(19) United States
(12) Patent Application Publication
(10) Pub. No.: US 2019/0233797 A1
(43) Pub. Date: Aug. 1, 2019

(54) HLA CLASS I-DEFICIENT NK-92 CELLS WITH DECREASED IMMUNOGENICITY

(71) Applicant: NANTKWEST, INC., San Diego, CA (US)

(72) Inventors: Francisco Navarro, Brookline, MA (US); Hans Klingemann, Boston, MA (US)

(73) Assignee: NANTKWEST, INC., San Diego, CA (US)

(21) Appl. No.: 16/337,996

(22) PCT Filed: Sep. 29, 2017
(86) PCT No.: PCT/US2017/054542

§ 371 Date: Mar. 29, 2019

Related U.S. Application Data
(60) Provisional application No. 62/401,653, filed on Sep. 29, 2016.

(51) Int. Cl. C12N 5/0783; C07K 14/74; A61P 35/00

(52) U.S. Cl. CPC C12N 5/0646; C12N 2510/00; A61P 35/00; C07K 14/70539

(57) ABSTRACT

Described herein are modified NK-92 cells comprising a genetic alteration to decrease beta-2-microglobulin (B2M) expression in NK-92 cells to reduce the levels of HLA class I expression; methods of generating such cells; and methods of treating a subject, e.g., that has cancer, with the B2M-modified NK-92 cells.

### CROSS-REFERENCE TO RELATED APPLICATIONS

This application claims priority benefit of U.S. provisional application No. 62/401,653, filed Sep. 29, 2016, which application is herein incorporated by reference.

### BACKGROUND OF THE INVENTION

Cell-based immunotherapies are a powerful tool for the treatment of cancer. Early success in the treatment of patients with lymphoid malignancies, using engineered primary T cells expressing chimeric antigen receptors (CAR-T cells), has propelled this field to the forefront of cancer immunotherapy. In addition to CAR-T cells, immunotherapies based on the use of NK cells are also being developed.

NK-92 is a cytolytic cancer cell line which was discovered in the blood of a subject suffering from a non-Hodgkins lymphoma and then immortalized ex vivo. NK-92 cells are derived from NK cells, but lack the major inhibitory receptors that are displayed by normal NK cells, while retaining the majority of the activating receptors. NK-92 cells do not, however, attack normal cells nor do they elicit an unacceptable immune rejection response in humans. Characterization of the NK-92 cell line is disclosed in WO 1998/49268 and U.S. Patent Application Publication No. 2002-0068044. NK-92 cells have also been evaluated as a potential therapeutic agent in the treatment of certain cancers.

### BRIEF SUMMARY OF ASPECTS OF THE INVENTION

The present invention provides modified NK-92 cells having decreased HLA class I expression, methods of producing such cells and methods of employing the modified NK-92 cells to treat a disease, e.g. cancer.

In one aspect, the disclosure thus provides a beta-2 microglobin (B2M)-modified NK-92 cell comprising a B2M-targeted alteration that inhibits expression of beta-2 microglobulin. In some embodiments, the beta-2 microglobulin gene of the B2M-modified NK-92 cell is genetically altered to inhibit expression of B2M. In some embodiments, the B2M-modified NK-92 cells comprising one or more interfering RNAs that target B2M and inhibit its expression. In some embodiments, the amount of beta-2-microglobulin expressed by the B2M-modified NK-92 cell is decreased by at least 20%, at least 30%, at least 50%, at least 60%, or at least 80% as compared to an NK-92 cells that do not have the beta-2-microglobulin-targeted alteration. In some embodiments, the B2M-modified NK-92 cell of claim 1 is produced by knocking down or knocking out beta-2 microglobulin in a an NK-92 cell, e.g., using CRISPR. In some embodiments, the cell is produced by knocking out beta-2 microglobulin in an NK-92 cell, e.g., using CRISPR. In some embodiments, the NK-92 cell is additionally modified to express a single chain trimer comprising an HLA-E binding peptide, B2M, and HLA-E heavy chain. In some embodiments, the single chain trimer comprises a B2M (β2 microglobulin) signal peptide, a Cw*03 leader peptide, e.g., a Cw*0304 leader peptide, a mature B2M polypeptide and a mature HLA-E polypeptide. In some embodiments, the Cw*03 leader peptide is linked to the mature B2M polypeptide by a flexible linker and/or the mature B2M polypeptide is linked to the mature HLA-E polypeptide by a flexible linker. One or both flexible linkers can comprise Gly and Ser. In some embodiments, the HLA-E heavy chain comprises a mature HLA-E^{G} amino acid sequence. In some embodiments, the single chain trimer comprises the amino acid sequence of SEQ ID NO:18.

In some embodiments, a B2M-modified NK-92 cell, e.g., as described herein and in the precending paragraph, expresses at least one Fc receptor or at least one chimeric antigen receptor (CAR). In some embodiments, a B2M-modified NK-92 cell, e.g., as described herein and in the precending paragraph, expresses at least one Fc receptor and at least one CAR on the cell surface. In some embodiments, the Fc receptor is CD16. In some embodiments, the CD16 polypeptide is a human CD16 polypeptide that has a valine at position 158 of the mature form, which corresponds to position 176 of the human CD16 sequence that includes the native signal peptide. In some embodiments, the at least one Fc receptor comprises a polynucleotide sequence encoding a polypeptide having at least 90% sequence identity to the amino acid sequence of SEQ ID NO:5 and comprises a valine at the position corresponding to position 158 of SEQ ID NO:5. In some embodiments, the Fc receptor is FcγRIII. In some embodiments, the CAR comprises a cytoplasmic domain of FcεRIγ. In some embodiments, the CAR targets a tumor-associated antigen. In some embodiments, B2M-modified NK-92 cell is modified to further express a cytokine. In some embodiments, the cytokine is interleukin-2 or a variant thereof. In some embodiments, the cytokine is targeted to the endoplasmic reticulum.

In another aspect, the disclosure provides a method for producing an NK-92 cell that expresses decreased levels of beta-2 microglobulin relative to a control NK-92 cell that is not genetically modified to decrease levels of beta-2 microglobulin, the method comprising genetically modifying beta-2 microglobulin expression in the NK-92 cell. In some embodiments, the step of genetically modifying beta-2 microglobulin expression comprises contacting a NK-92 cell to be modified with an interfering RNA targeting beta-2 microglobulin. In some embodiments, the interfering RNA targeting beta-2 microglobulin is an siRNA, an shRNA, a microRNA, or a single stranded interfering RNA.

In some embodiments, the step of genetically modifying beta-2 microglobulin expression comprises modifying the beta-2 microglobulin gene with a zinc finger nuclease (ZFN), a Tale-effector domain nuclease (TALEN), or a CRIPSR/Cas system. In some embodiments, genetically modifying the beta-2 microglobulin gene expression comprises: i) introducing a clustered regularly interspaced short palindromic repeat-associated (Cas) protein into the NK-92 cell and ii) introducing one or more ribonucleic acids in the NK-92 cell to be modified, wherein the ribonucleic acids direct the Cas protein to hybridize to a target motif of the beta-2 microglobulin sequence, and wherein the target motif is cleaved. In some embodiments, the Cas protein is introduced into the NK-92 cell in protein form. In some embodiments, the Cas protein is introduced into the NK-92 cell by introducing a Cas nucleic acid coding sequence. In some embodiments, the Cas protein is Cas9. In some embodiments, the target motif is a 20 nucleotide DNA sequence. In some embodiments, the target motif is in the first exon of beta 2 microglobulin gene. In some embodiments, the one or more ribonucleic acids are selected from the group consisting of SEQ ID NOs. 1-4.

In a further aspect, the disclosure provides a composition comprising a plurality of the B2M-modified NK-92 cells disclosed above. In some embodiments, the composition also comprises a physiologically acceptable excipient.

In an additional aspect, the disclosure provides a modified NK-92 cell line comprising a plurality of any of the B2M-modified NK-92 cells disclosed above. In some embodiments, the cells of the cell line undergo less than 10 population doublings. In some embodiments, the cells of the cell line are cultured in media containing less than 10 U/ml of IL-2.

In another aspect, the disclosure provides a method of treating cancer in a patient in need thereof, the method comprising administering to the patient a therapeutically effective amount of any of the B2M-modified NK-92 cell lines described above, thereby treating the cancer. In some embodiments, the method further comprising administering an antibody. In some embodiments, about 1×10⁸ to about 1×10¹¹ cells per m² of body surface area of the patient are administered to the patient.

In a further aspect, the disclosure provides a kit for treating cancer, wherein the kit comprises (a) any of the B2M-modified NK-92 cell compositions, or cell lines, as disclosed above, and (b) instructions for use. In some embodiments, the kit further comprises a physiologically acceptable excipient.

The foregoing summary and the following detailed description are exemplary and explanatory and are intended to provide further explanation of the invention as claimed. Other objects, advantages and novel features will be readily apparent to those skilled in the art from the following detailed description of the invention.

Illustrative embodiments of the invention include, but are not limited to, the following:

### Embodiment 1

A beta-2-microglobulin-modified (B2M-modified) NK-92 cell comprising a beta-2 microglobulin-targeted genetic modification to inhibit expression of beta-2 microglobulin.

### Embodiment 2

The B2M-modified NK-92 cell of Embodiment 1, wherein the cell is produced by knocking down or knocking out beta-2 microglobulin in an NK-92 cell.

### Embodiment 3

The B2M-modified NK-92 cell of Embodiment 2, comprising an interfering RNA that targets B2M and inhibits its expression.

### Embodiment 4

The B2M-modified NK-92 cell of any one of Embodiments 1 to 3, wherein the amount of beta-2-microglobulin expressed by the cell is decreased by at least 50%, at least 60%, at least, 70%, or at least 80% as compared to an NK-92 cells that do not have the beta-2-microglobulin-targeted alteration.

### Embodiment 5

The B2M-modified NK-92 cell of Embodiment 1, wherein the cell is produced by knocking out beta-2 microglobulin in an NK-92 cell.

### Embodiment 6

The B2M-modified NK-92 cell of any one of Embodiments 1 to 5, wherein the cell is modified to express a single chain trimer comprising an HLA-E binding peptide, B2M, and HLA-E heavy chain.

### Embodiment 7

The B2M-modified NK-92 cell of Embodiment 6, wherein the single chain trimer comprises a B2M ((32 microglobulin) signal peptide, a Cw*0304 leader peptide, a mature B2M polypeptide and a mature HLA-E polypeptide.

### Embodiment 8

The B2M-modified NK-92 cell of Embodiment 7, wherein the Cw*0304 leader peptide is linked to the mature B2M polypeptide by a flexible linker and/or the mature B2M polypeptide is linked to the mature HLA-E polypeptide by a flexible linker.

### Embodiment 9

The B2M-modified NK-92 cell of Embodiment 8, wherein the flexible linker that links the C2*0304 leader peptide to the mature B2M polypeptide and/or the flexible linker that link the mature B2M polypeptide to the mature HLA-E polypeptide comprises Gly and Ser.

### Embodiment 10

The B2M-modified NK-92 cell of any one of Embodiments 6 to 9, wherein the HLA-E heavy chain comprises a mature HLA-EG amino acid sequence.

### Embodiment 11

The B2M-modified NK-92 cell of any one of Embodiments 6 to 10, wherein the single chain trimer comprises the amino acid sequence of SEQ ID NO:18.

### Embodiment 12

The B2M-modified NK-92 cell of any one of Embodiments 1 to 11, wherein the B2M-modified NK cell expresses at least one Fc receptor on the cell surface or at least one chimeric antigen receptor (CAR) on the cell surface; or at least one Fc receptor and at least one CAR on the cell surface.

### Embodiment 13

The B2M-modified NK-92 cell of Embodiment 12, wherein the at least one Fc receptor is a human CD16 polypeptide having a valine at position 158 of the mature form of the CD16 polypeptide.

### Embodiment 14

The B2M-modified NK-92 cell of Embodiment 12, wherein the at least one Fc receptor comprises a polynucleotide sequence encoding a polypeptide having at least 90% sequence identity to the amino acid sequence of SEQ ID NO:5 and comprises a valine at a position corresponding to position 158 of SEQ ID NO:5.

### Embodiment 15

The B2M-modified NK-92 cell of Embodiment 12, wherein the at least one Fc receptor is FcγRIII.

### Embodiment 16

The B2M-modified NK-92 cell of any one of Embodiments 12 to 15, wherein the CAR comprises a cytoplasmic domain of FcεRIγ.

### Embodiment 17

The B2M-modified NK-92 cell of any one of Embodiments 12 to 16, wherein the CAR targets a tumor-associated antigen.

### Embodiment 18

The B2M-modified NK-92 cell of any one of Embodiments 1 to 17, wherein the cell further expresses a cytokine.

### Embodiment 19

The B2M-modified NK-92 cell of Embodiment 18, wherein the cytokine is interleukin-2 or a variant thereof.

### Embodiment 20

The B2M-modified NK-92 cell of Embodiment 19, wherein the cytokine is targeted to the endoplasmic reticulum.

### Embodiment 21

A composition comprising a plurality of cells of any one of Embodiments 1 to 20.

### Embodiment 22

The composition of Embodiment 21, further comprising a physiologically suitable excipient.

### Embodiment 23

A modified NK-92 cell line comprising a plurality of modified NK-92 cells of any one of Embodiments 1 to 20.

### Embodiment 24

The cell line of Embodiment 23, wherein the cells undergo less than 10 population doublings.

### Embodiment 25

The cell line of Embodiment 23, wherein the cells are cultured in media containing less than 10 U/ml of IL-2.

### Embodiment 26

A method of treating cancer in a patient in need thereof, the method comprising administering to the patient a therapeutically effective amount of the cell line of embodiment 23, thereby treating the cancer.

### Embodiment 27

The method of Embodiment 26, wherein the method further comprising administering an antibody.

### Embodiment 28

The method of Embodiment 26 or 27, wherein about 1×10⁸ to about 1×10¹¹ cells per m² of body surface area of the patient are administered to the patient.

### Embodiment 29

A method for producing an NK-92 cell that expresses decreased levels of beta-2 microglobulin relative to a control NK-92 cell, the method comprising genetically modifying the NK-92 cell to inhibit beta-2 microglobulin expression.

### Embodiment 30

The method of Embodiment 29, wherein the step of genetically modifying beta-2 microglobulin expression comprises modifying the beta-2 microglobulin gene with a zinc finger nuclease (ZFN), a Tale-effector domain nuclease (TALEN), or a CRIPSR/Cas system to eliminate or reduce expression of the beta-2 microglobulin gene.

### Embodiment 31

The method of Embodiment 30, wherein the step of genetically modifying beta-2 microglobulin expression comprises modifying the beta-2 microglobulin gene with a CRIPSR/Cas system to eliminate or reduce expression of the beta-2 microglobulin gene.

### Embodiment 32

The method of Embodiment 29, wherein the step of genetically modifying beta-2 microglobulin expression comprises contacting a NK-92 cell to be modified with an interfering RNA targeting beta-2 microglobulin.

### Embodiment 33

The method of Embodiment 32, wherein the interfering RNA targeting beta-2 microglobulin is an siRNA, an shRNA, a microRNA, or a single stranded interfering RNA.

### Embodiment 34

method of any one of Embodiments 29 to 33, wherein the amount of beta-2-microglobulin expressed by the cell is decreased by at least 50%, at least 60%, at least, 70%, or at least 80% as compared to an NK-92 cells that do not have the beta-2-microglobulin-targeted alteration

### Embodiment 35

The method of Embodiment 29, wherein genetically modifying the beta-2 microglobulin gene expression comprises:

i) introducing a clustered regularly interspaced short palindromic repeat-associated (Cas) protein into the NK-92 cell and
ii) introducing one or more ribonucleic acids in the NK-92 cell to be modified, wherein the ribonucleic acids direct the Cas protein to hybridize to a target motif of the beta-2 microglobulin sequence, and wherein the target motif is cleaved.

### Embodiments 36

The method of Embodiment 35, wherein the Cas protein is introduced into the NK-92 cell in protein form.

### Embodiment 37

The method of Embodiment 35, wherein the Cas protein is introduced into the NK-92 cell by introducing a Cas-encoding polynucleotide into the NK-92 cells.

### Embodiment 38

The method of any one of Embodiments 35 to 37, wherein the Cas protein is Cas9.

### Embodiment 39

The method of any one of Embodiments 35 to 38, wherein the target motif is in the first exon of beta 2 microglobulin gene.

### Embodiment 40

The method of Embodiment 39, wherein the target motif is a 20 nucleotide DNA sequence.

### Embodiment 41

The method of any one of Embodiments 35 to 40, wherein the one or more ribonucleic acids are selected from the group consisting of SEQ ID NOs. 1-4.

### BRIEF DESCRIPTION OF THE DRAWINGS

FIG. 1 provides illustrative data showing an analysis of immunogenicity of NK-92 cells in mixed lymphocyte reactions. Autologous or unstimulated PBMCs were used as negative controls, while Staphylococcal enterotoxin B superantigen (SEB) was used as positive control for proliferation. NK-92 cells were irradiated at 6,000 rad and used to stimulate 500,000 PBMCs from 9 healthy controls at 1:1 ratio. IFN-g production (top) and proliferation (bottom) of CD4+ (left) or CD8+ (right) T cells were measured after 1 and 5 days, respectively.

FIG. 2 provides illustrative data showing that Cas9-NK-92 and Cas9-haNK cell lines expressed high levels of Cas9 protein.

FIG. 3 provides illustrative flow cytometry data analyzing beta-2-microglobulin (B2M) expression in untransfected Cas9-NK-92 cells or cells transfected with 10 μg of in vitro transcribed B2M sgRNA-1 RNA.

FIG. 4 panels A and B provide illustrative flow cytometry data showing analysis of B2M and HLA class I expression in wild type and B2M-KO Cas9-NK-92 cells. The results demonstrated that B2M-KO Cas9-NK-92 cells were deficient in classical HLA class I (A, B, C) and non-classical HLA-E expression.

FIG. 5 provides illustrative data showing that B2M-KO NK-92 cells are susceptible to lysis by allogeneic NK cells. The ability of freshly isolated (left) or activated (right) primary NK cells to lyse either parental (NK-92 and Cas9-NK-92) or B2M-KO (clones #27 and #37) NK-92 cells was evaluated in a 4 hour cytotoxicity assay at different effector to target (E:T) ratios. K562, HLA-I deficient erythroleukemia cells highly susceptible to NK cell lysis, are included as positive control. “n” indicates number of donors tested.

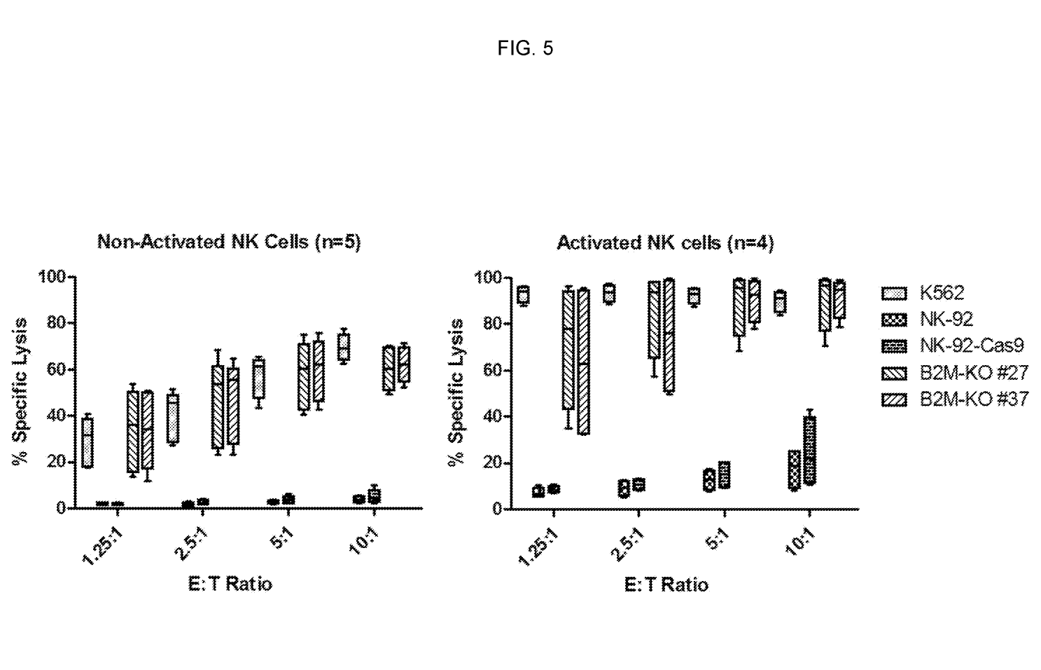

FIG. 6 shows a schematic of an illustrative HLA-E-SCT (single chain trimer) molecule. The chimeric HLA-E-SCT molecule is composed of B2M (β2 microglobulin) signal peptide, Cw*03 peptide, (G₄S)₃ linker, mature B2M chain, (G₄S)₄ linker, and mature HLA-E chain.

FIG. 7 provides illustrative data showing efficient HLA-E-SCT expression in HLA-I deficient NK-92 cells. Flow cytometry analysis of B2M, HLA-I (A, B, and C), and HLA-E expression in parental B2M-KO NK-92 and and HLA-E-SCT expressing B2M-KO NK-92 cells.

FIG. 8 provides illustrative data showing that enforced HLA-E-SCT expression in HLA-I deficient NK-92 cells confers partial protection against lysis by allogeneic NK cells. Susceptibility of parental (NK-92 and NK-92-Cas9), B2M-KO (clones #27 and #37), and HLA-E-SCT expressing B2M-KO NK-92 cells to lysis by allogeneic NK cells was evaluated in a 4 hour cytotoxicity assay at different effector to target (E:T) ratios, using either freshly isolated (left) or activated (right) primary NK cells. Parental and HLA-E-SCT expressing K562 cells are included as reference. “n” indicates number of donors tested.

FIG. 9 provides illustrative data showing that HLA-I deficient NK-92 cells are resistant to lysis by NK-92 specific allogeneic CD8+ T cells. The ability of NK-92 specific allogeneic CD8+ T cells to lyse either parental (NK-92 and NK-92-Cas9), B2M-KO (clones #27 and #37), or HLA-E-SCT expressing B2M-KO NK-92 cells was evaluated in a 4 hour cytotoxicity assay at two different effector to target (E:T) ratios. 1B9 (left) and 2H6 (right) correspond to two different oligoclonal CD8+ T cell populations generated against parental NK-92 cells.

### DETAILED DESCRIPTION OF THE INVENTION

In one aspect, the invention provides methods and compositions to reduce the immunogenicity of therapeutic NK-92 cells that are administered for treatment of a disorder and avoid undesired consequences that administered NK-92 cells become a target for the patient's T cells. The present invention thus provides B2M-modified NK-92 cells having decreased HLA class I expression and methods of producing such cells. B2M-modified NK-92 cells in accordance with the present disclosure have a B2M-targeted alteration in the NK-92 cells. Such modifications minimize the risk of NK-92 cells being attacked by a recipient's own immune system and thus increase the efficiency of NK-92 cell therapy.

### Terminology

Unless defined otherwise, all technical and scientific terms used herein have the same meaning as commonly understood by one of ordinary skill in the art to which this invention belongs.

In this specification and in the claims that follow, reference will be made to a number of terms that shall be defined to have the following meanings:

The terminology used herein is for the purpose of describing particular embodiments only and is not intended to be limiting of the invention. As used herein, the singular forms “a”, “an” and “the” are intended to include the plural forms as well, unless the context clearly indicates otherwise.

As used herein, the terms “about” and “approximately,” when used to modify an amount specified in a numeric value or range, indicate that the numeric value as well as reasonable deviations from the value known to the skilled person in the art, for example ±20%, ±10%, or ±5%, are within the intended meaning of the recited value.

The term “comprising” is intended to mean that the compositions and methods include the recited elements, but do not exclude others. “Consisting essentially of” when used to define compositions and methods, refers to the specified materials or steps and those that do not materially affect the basic and novel characteristic(s) of the claimed invention. “Consisting of” shall mean excluding more than trace amounts of other ingredients and substantial method steps recited. Embodiments defined by each of these transition terms are within the scope of this invention.

The term “natural killer (NK) cells” refers to cells of the immune system that kill target cells in the absence of a specific antigenic stimulus, and without restriction according to MHC class. Target cells may be tumor cells or cells harboring viruses. NK cells are characterized by the presence of CD56 and the absence of CD3 surface markers.

The term “NK-92 cells”, which are also referred to as “aNK cells” in the examples section of this disclosure, refer to the NK cell line, NK-92, which was originally obtained from a patient having non-Hodgkin's lymphoma. For purposes of this invention and unless indicated otherwise, the term “NK-92” is intended to refer to the original NK-92 cell lines as well as NK-92 cell lines, clones of NK-92 cells, and NK-92 cells that have been modified (e.g., by introduction of exogenous genes. NK92 cells and exemplary and non-limiting modifications thereof are described in U.S. Pat. Nos. 7,618,817, 8,034,332, and 8,313,943, and US Patent Application Publication No. 2013/0040386, all of which are incorporated herein by reference in their entireties, and include wild type NK92, NK92-CD16, NK92-CD16-γ, NK92-CD16-ζ, NK92-CD16(F176V), NK92MI, and NK92CI. NK92 cells are known and readily available to a person of ordinary skill in the art from NantKwest, Inc.

The term “B2M-targeted alteration” refers to a change to the structure or properties of DNA or RNA of B2M in a NK-92 cell, for example, knocking out or knocking down B2M expression, which leads to a decrease in the level of B2M protein. Thus, a B2M-targeted alteration can target the B2M gene or a B2M gene transcript. An example of a human B2M protein sequence (human B2M precursor) is available under accession number NP_004039. Human B2M is located on chromosome 15 and is mapped to position 15q21-q22.2. The Unigene accession number is Hs.534255 and is located at 44.71-44.72 Mb of chromosome 15 according to the Genome Reference Consortium Human Build 38 patch release 7 (GRCh38.p7), Annotation Release 108. The term “B2M” also encompasses allelic variants of the exemplary references sequence that are encoded by a gene at the B2M chromosomal locus.

The term “B2M-modified NK-92 cell” refers to an NK-92 cell that has a B2M-targeted alteration that results in a decrease in amount of B2M expression. The genetically modified NK-92 cells may further comprise a vector that encodes HLA-E and/or other transgenes, such as an a Fc receptor, chimeric antigen receptor (CAR), IL-2, or a suicide gene.

The term “B2M-unmodified NK-92 cells” refers to the NK-92 cells that do not have a B2M targeted alteration that decreased B2M expression.

The term “non-irradiated NK-92 cells” refers to NK-92 cells that have not been irradiated. Irradiation renders the cells incapable of growth and proliferation. In some embodiments, NK-92 cells for administration may be irradiated at a treatment facility or some other point prior to treatment of a patient, as in some embodiments, the time between irradiation and infusion is no longer than four hours in order to preserve optimal activity. Alternatively, NK-92 cells may be inactivated by another mechanism.

As used to describe the present invention, “inactivation” of the NK-92 cells renders them incapable of growth. Inactivation may also relate to the death of the NK-92 cells. It is envisioned that the NK-92 cells may be inactivated after they have effectively purged an ex vivo sample of cells related to a pathology in a therapeutic application, or after they have resided within the body of a mammal a sufficient period of time to effectively kill many or all target cells residing within the body. Inactivation may be induced, by way of non-limiting example, by administering an inactivating agent to which the NK-92 cells are sensitive.

As used to describe the present invention, the terms “cytotoxic” and “cytolytic”, when used to describe the activity of effector cells such as NK cells, are intended to be synonymous. In general, cytotoxic activity relates to killing of target cells by any of a variety of biological, biochemical, or biophysical mechanisms. Cytolysis refers more specifically to activity in which the effector lyses the plasma membrane of the target cell, thereby destroying its physical integrity. This results in the killing of the target cell. Without wishing to be bound by theory, it is believed that the cytotoxic effect of NK cells is due to cytolysis.

The term “kill” with respect to a cell/cell population is directed to include any type of manipulation that will lead to the death of that cell/cell population.

The term “Fc receptor” refers to a protein found on the surface of certain cells (e.g., natural killer cells) that contribute to the protective functions of the immune cells by binding to part of an antibody known as the Fc region. Binding of the Fc region of an antibody to the Fc receptor (FcR) of a cell stimulates phagocytic or cytotoxic activity of a cell via antibody-mediated phagocytosis or antibody-dependent cell-mediated cytotoxicity (ADCC). FcRs are classified based on the type of antibody they recognize. For example, Fc-gamma receptors (FCγR) bind to the IgG class of antibodies. FCγRIII-A (also called CD16) is a low affinity Fc receptor bind to IgG antibodies and activate ADCC. FCγRIII-A are typically found on NK cells. A representative polynucleotide sequence encoding a native form of CD16 is shown in SEQ ID NO:5.

The terms “polynucleotide”, “nucleic acid” and “oligonucleotide” are used interchangeably and refer to a polymeric form of nucleotides of any length, either deoxyribonucleotides or ribonucleotides or analogs thereof. Polynucleotides can have any three dimensional structure and may perform any function, known or unknown. The following are non limiting examples of polynucleotides: a gene or gene fragment (for example, a probe, primer, EST or SAGE tag), exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes and primers. A polynucleotide can comprise modified nucleotides, such as methylated nucleotides and nucleotide analogs. If present, modifications to the nucleotide structure can be imparted before or after assembly of the polynucleotide. The sequence of nucleotides can be interrupted by non nucleotide components. A polynucleotide can be further modified after polymerization, such as by conjugation with a labeling component. The term also refers to both double and single stranded molecules. Unless otherwise specified or required, any embodiment of this invention that is a polynucleotide encompasses both the double stranded form and each of two complementary single stranded forms known or predicted to make up the double stranded form. Unless indicated otherwise, nucleic acid sequences are shown 5′ to 3′.

A polynucleotide is composed of a specific sequence of four nucleotide bases, e.g., the naturally occurring bases adenine (A); cytosine (C); guanine (G); thymine (T); and uracil (U) for thymine when the polynucleotide is RNA. Thus, the term “polynucleotide sequence” is the alphabetical representation of a polynucleotide molecule.

The term “percent identity” refers to sequence identity between two peptides or between two nucleic acid molecules. Percent identity can be determined by comparing a position in each sequence which may be aligned for purposes of comparison. When a position in the compared sequence is occupied by the same base or amino acid, then the molecules are identical at that position. As used herein, the phrase “variant” nucleotide sequence,” or “variant” amino acid sequence refers to sequences characterized by identity, at the nucleotide level or amino acid level, of at least a specified percentage. Variant nucleotide sequences include those sequences coding for naturally occurring allelic variants and mutations of the nucleotide sequences set forth herein. Variant nucleotide sequences include nucleotide sequences encoding for a protein of a mammalian species other than humans. Variant amino acid sequences include those amino acid sequences which contain conservative amino acid substitutions and which polypeptides have the same binding and/or activity. In some embodiments, a variant nucleotide or amino acid sequence has at least 60% or greater identity, for example at least 70%, or at least 80%, at least 85% or greater, identity with a reference sequence. In some embodiments, a variant nucleotide or amino acid sequence has at least 90%, 91%, 92%, 93%, 94%, 95%, 96%, 97%, 98% or 99% density with a reference sequence. In some embodiments, variant amino acid sequence has no more than 15, nor more than 10, nor more than 5 or no more than 3 conservative amino acid substitutions. Percent identity can be determined by known algorithms, for example, the Gap program (Wisconsin Sequence Analysis Package, Version 8 for UNIX, Genetics Computer Group, University Research Park, Madison Wis.), using default settings, which uses the algorithm of Smith and Waterman (Adv. Appl. Math., 1981, 2, 482-489).

The terms “corresponding to,” or “determined with reference to,” when used in the context of the identification of a given amino acid residue in a polypeptide sequence, refers to the position of the residue of a specified reference sequence when the given amino acid sequence is maximally aligned and compared to the reference sequence.

The term “express” refers to the production of a gene product, which may be an RNA or protein.

The term “cytokine” or “cytokines” refers to the general class of biological molecules which effect cells of the immune system. Exemplary cytokines for use in practicing the invention include but are not limited to interferons and interleukins (IL), in particular IL-2, IL-12, IL-15, IL-18 and IL-21. In preferred embodiments, the cytokine is IL-2.

The term “vector” refers to a non-chromosomal nucleic acid comprising an intact replicon such that the vector may be replicated when placed within a permissive cell, for example by a process of transformation. A vector may replicate in one cell type, such as bacteria, but have limited ability to replicate in another cell, such as mammalian cells. Vectors may be viral or non-viral. Exemplary non-viral vectors for delivering nucleic acid include naked DNA; DNA complexed with cationic lipids, alone or in combination with cationic polymers; anionic and cationic liposomes; DNA-protein complexes and particles comprising DNA condensed with cationic polymers such as heterogeneous polylysine, defined-length oligopeptides, and polyethylene imine, in some cases contained in liposomes; and the use of ternary complexes comprising a virus and polylysine-DNA.

The term “target motif” refers to a nucleic acid sequence that defines a portion of a nucleic acid to which a binding molecule will bind, provided sufficient conditions for binding exist.

The term “interfering RNA” refers to an RNA nucleic acid molecule which is double stranded or single stranded and is capable of effecting the induction of an RNA interference mechanism directed to knocking down the expression of a target gene.

The terms “patient,” “subject,” “individual,” and the like are used interchangeably herein, and refer to any animal, or cells thereof whether in vitro or in situ, amenable to the methods described herein. In certain non-limiting embodiments, the patient, subject or individual is a human.

The term “recipient,” refers a patient who is administered NK-92 cells, whether modified or unmodified, during treatment.

The term “treating” or “treatment” covers the treatment of a disease or disorder described herein, in a subject, such as a human, and includes: (i) inhibiting a disease or disorder, i.e., arresting its development; (ii) relieving a disease or disorder, i.e., causing regression of the disorder; (iii) slowing progression of the disorder; and/or (iv) inhibiting, relieving, or slowing progression of one or more symptoms of the disease or disorder. The term “administering” or “administration” of a monoclonal antibody or a natural killer cell to a subject includes any route of introducing or delivering the antibody or cells to perform the intended function. Administration can be carried out by any route suitable for the delivery of the cells or monoclonal antibody. Thus, delivery routes can include intravenous, intramuscular, intraperitoneal, or subcutaneous deliver. In some embodiments NK-92 cells are administered directly to the tumor, e.g., by injection into the tumor.

The term “contacting” (i.e., contacting a polynucleotide sequence with a clustered regularly interspaced short palindromic repeats-associated (Cas) protein and/or ribonucleic acids) is intended to include incubating the Cas protein and/or the ribonucleic acids in the cell together in vitro (e.g., adding the Cas protein or nucleic acid encoding the Cas protein to cells in culture). In some embodiments, the term “contacting” is not intended to include the in vivo exposure of cells to the Cas protein and/or ribonucleic acids as disclosed herein that may occur naturally in a microorganism (i.e., bacteria). The step of contacting a target polynucleotide sequence with a Cas protein and/or ribonucleic acids as disclosed herein can be conducted in any suitable manner. For example, the cells may be treated in adherent culture, or in suspension culture. It is understood that the cells contacted with a Cas protein and/or ribonucleic acids as disclosed herein can also be simultaneously or subsequently contacted with another agent, such as a growth factor or other differentiation agent or environments to stabilize the cells, or to differentiate the cells further.

As used herein, the term “knock out” includes deleting all or a portion of a target polynucleotide sequence in a way that interferes with the function of the target polynucleotide sequence such that an RNA and/or protein product encoded by the target polynucleotide is not expressed. For example, a knock out can be achieved by altering a target polynucleotide sequence by inducing an indel in the target polynucleotide sequence in a functional domain of the target polynucleotide sequence (e.g., a DNA binding domain). Those skilled in the art will readily appreciate how to use various genetic approaches, e.g., CRISPR/Cas systems, ZFN, TALEN, TgAgo, to knock out a target polynucleotide sequence or a portion thereof based upon the details described herein.

As used herein, the term “knock down” refers to a measurable reduction in expression of a target mRNA or the corresponding protein in a genetically modified cell as compared with the expression of the target mRNA or the corresponding protein in a counterpart cell that does not contain the genetic modification to reduce expression. Those skilled in the art will readily appreciate how to use various genetic approaches, e.g., siRNA, shRNA, microRNA, antisense RNA, or other RNA-mediated inhibition techniques, to knock down a target polynucleotide sequence or a portion thereof based upon the details described herein.

The terms “decrease” or “reduced” are used interchangeably herein to refer to a decrease by at least 10% as compared to a reference level, e.g., a counterpart cell that does not have the genetic modification to reduce B2M expression. In some embodiments, expression is decreased by at least about 20%, or at least about 30%, or at least about 40%, or at least about 50%, or at least about 60%, or at least about 70%, or at least about 80%, or at least about 90% or up to and including a 100% decrease (i.e. absent level as compared to a reference sample), or any decrease between 10-100% as compared to a reference level.

The term “cancer” refers to all types of cancer, neoplasm, or malignant tumors found in mammals, including leukemia, carcinomas and sarcomas. Exemplary cancers include cancer of the brain, breast, cervix, colon, head & neck, liver, kidney, lung, non-small cell lung, melanoma, mesothelioma, ovary, sarcoma, stomach, uterus and medulloblastoma. Additional examples include, Hodgkin's Disease, Non-Hodgkin's Lymphoma, multiple myeloma, neuroblastoma, ovarian cancer, rhabdomyosarcoma, primary thrombocytosis, primary macroglobulinemia, primary brain tumors, cancer, malignant pancreatic insulanoma, malignant carcinoid, urinary bladder cancer, premalignant skin lesions, testicular cancer, lymphomas, thyroid cancer, neuroblastoma, esophageal cancer, genitourinary tract cancer, malignant hypercalcemia, endometrial cancer, adrenal cortical cancer, neoplasms of the endocrine and exocrine pancreas, and prostate cancer.

### NK-92 Cells

The NK-92 cell line is a unique cell line that was discovered to proliferate in the presence of interleukin 2 (IL-2). Gong et al., Leukemia 8:652-658 (1994). These cells have high cytolytic activity against a variety of cancers. The NK-92 cell line is a homogeneous cancerous NK cell population having broad anti-tumor cytotoxicity with predictable yield after expansion. Phase I clinical trials have confirmed its safety profile.

The NK-92 cell line is found to exhibit the CD56^{bright}, CD2, CD7, CD11a, CD28, CD45, and CD54 surface markers. It furthermore does not display the CD1, CD3, CD4, CD5, CD8, CD10, CD14, CD16, CD19, CD20, CD23, and CD34 markers. Growth of NK-92 cells in culture is dependent upon the presence of recombinant interleukin 2 (rIL-2), with a dose as low as 1 IU/mL being sufficient to maintain proliferation. IL-7 and IL-12 do not support long-term growth, nor do other cytokines tested, including IL-1α, IL-6, tumor necrosis factor α, interferon α, and interferon γ. NK-92 has high cytotoxicity even at a low effector:target (E:T) ratio of 1:1. Gong, et al., supra.

Heretofore, studies on endogenous NK cells have indicated that IL-2 (1000 IU/mL) is important for NK cell activation during shipment, but that the cells need not be maintained at 37° C. and 5% carbon dioxide. Koepsell, et al., Transfusion 53:398-403 (2013).

### HLA Class I

The human leukocyte antigen (HLA) system is a gene complex encoding the major histocompatibility complex (MHC) proteins in humans. The HLA class I proteins all have a long alpha chain and a short beta chain, B2M. Little HLA class I can be expressed in the absence of B2M and the expression of B2M is required for HLA class I proteins to present peptides from inside the cell. The present disclosure provides a B2M-modified NK-92 cell that expresses decreased amount of B2M as compared to unB2M-modified NK-92 cells. Thus, these cells avoid the immune surveillance and attack by cytotoxic T cells. In one embodiment, the B2M is SEQ ID NO: 6.

The instant disclosure provides a B2M-modified NK-92 cell comprising a B2M-targeted alteration that inhibits expression of B2M. In some embodiments, the B2M-modified NK-92 cell is generated by CRISPR/Cas9-mediated genetic ablation of B2M. In some embodiments, the B2M-modified NK-92 cells are produced by knocking down B2M. The disclosure also provides methods for treating cancer in a patient in need thereof comprising administering to the patient a therapeutically effective amount of the cell line comprising the B2M-modified NK-92 cells.

### Knocking Out Beta 2 Microglobulin in NK-92 Cells

In some embodiments, the B2M-modified NK-92 cells comprising a B2M-targeted alteration are produced by knocking out B2M in NK-92 cells. Methods for knocking out a target gene expression include, but not limited to, a zinc finger nuclease (ZFN), a Tale-effector domain nuclease (TALEN), and CRIPSR/Cas system. Such methods typically comprise administering to the cell one or more polynucleotides encoding one or more nucleases such that the nuclease mediates modification of the endogenous gene, for example in the presence of one or more donor sequence, such that the donor is integrated into the endogenous gene targeted by the nuclease. Integration of one or more donor molecule(s) occurs via homology-directed repair (HDR) or by non-homologous end joining (NHEJ) associated repair. In certain embodiments, one or more pairs of nucleases are employed, which nucleases may be encoded by the same or different nucleic acids.

### CRISPR

In some embodiments, the knocking out or knocking down of B2M is performed using CRIPSR/Cas system. CRISPR/Cas system includes a Cas protein and at least one to two ribonucleic acids that are capable of directing the Cas protein to and hybridizing to a target motif in the B2M sequence. The Cas protein then cleaves the target motif and result in a double-strand break or a single-strand break results. Any CRISPR/Cas system that is capable of altering a target polynucleotide sequence in a cell can be used. In some embodiments, the CRISPR Cas system is a CRISPR type I system, in some embodiments, the CRISPR/Ca system is a CRISPR type II system. In some embodiments, the CRISPR/Cas system is a CRISPR type V system.

The Cas protein used in the invention can be a naturally occurring Cas protein or a functional derivative thereof. A “functional derivative” includes, but are not limited to, fragments of a native sequence and derivatives of a native sequence polypeptide and its fragments, provided that they have a biological activity in common with a corresponding native sequence polypeptide. A biological activity contemplated herein is the ability of the functional derivative to hydrolyze a DNA substrate into fragments. The term “derivative” encompasses both amino acid sequence variants of polypeptide, covalent modifications, and fusions thereof such as derivative Cas proteins. Suitable derivatives of a Cas polypeptide or a fragment thereof include but are not limited to mutants, fusions, covalent modifications of Cas protein or a fragment thereof.

In some embodiments, the Cas protein used in the invention is Cas9 or a functional derivative thereof. In some embodiments, the Cas9 protein is from Streptococcus pyogenes. Cas 9 contains 2 endonuclease domains, including an RuvC-like domain which cleaves target DNA that is noncomplementary to crRNA, and an HNH nuclease domain which cleave target DNA complementary to crRNA. The double-stranded endonuclease activity of Cas9 also requires that a short conserved sequence, (2-5 nucleotides), known as a protospacer-associated motif (PAM), follows immediately 3′- of a target motif in the target sequence.

In some embodiments, the Cas protein is introduced into the NK-92 cells in polypeptide form. In certain embodiments, the Cas proteins can be conjugated to or fused to a cell-penetrating polypeptide or cell-penetrating peptide that is well known in the art. Non-limiting examples of cell-penetrating peptides include those provided in Milletti F, Cell-penetrating peptides: classes, origin and current landscape. Drug Discov. Today 17: 850-860 (2012), the relevant disclosure of which is hereby incorporated by reference in its entirety. In some cases, an B2M-unmodified NK-92 cell is genetically engineered to produce the Cas protein.

In some embodiments, the target motif in the B2M gene, to which the Cas protein is directed by the guide RNAs, is 17 to 23 bp in length. In some embodiments, the target motif is at least 20 bp in length. In some embodiments, the target motif is a 20-nucleotide DNA sequence. In some embodiments, the target motif is a 20-nucleotide DNA sequence and immediately precedes a short conserved sequence known as a protospacer-associated motif (PAM), recognized by the Cas protein. In some embodiments, the PAM motif is an NGG motif. In some embodiments, the target motif of the B2M gene is within the first exon.

In some embodiments, the target motifs can be selected to minimize off-target effects of the CRISPR/Cas systems of the present invention. In some embodiments, the target motif is selected such that it contains at least two mismatches when compared with all other genomic nucleotide sequences in the cell. In some embodiments, the target motif is selected such that it contains at least one mismatch when compared with all other genomic nucleotide sequences in the cell. Those skilled in the art will appreciate that a variety of techniques can be used to select suitable target motifs for minimizing off-target effects (e.g., bioinformatics analyses).

The ribonucleic acids that are capable of directing the Cas protein to and hybridizing to a target motif in the B2M sequence are referred to as single guide RNA (“sgRNA”). The sgRNAs can be selected depending on the particular CRISPR/Cas system employed, and the sequence of the target polynucleotide, as will be appreciated by those skilled in the art. In some embodiments, the one to two ribonucleic acids can also be selected to minimize hybridization with nucleic acid sequences other than the target polynucleotide sequence. In some embodiments, the one to two ribonucleic acids hybridize to a target motif that contains at least two mismatches when compared with all other genomic nucleotide sequences in the cell. In some embodiments, the one to two ribonucleic acids hybridize to a target motif that contains at least one mismatch when compared with all other genomic nucleotide sequences in the cell. In some embodiments, the one to two ribonucleic acids are designed to hybridize to a target motif immediately adjacent to a deoxyribonucleic acid motif recognized by the Cas protein. In some embodiments, each of the one to two ribonucleic acids are designed to hybridize to target motifs immediately adjacent to deoxyribonucleic acid motifs recognized by the Cas protein which flank a mutant allele located between the target motifs. Guide RNAs can also be designed using software that are readily available, for example, at http://crispr.mit.edu. The one or more sgRNAs can be transfected into the NK-92 cells in which Cas protein is present by transfection, according to methods known in the art. In some embodiments, the sgRNAs are selected from the group consisting of SEQ ID NOs: 1-4.

Methods of using the CRISPR/Cas system to reduce gene expression are described in various publications, e.g., US. Pat. Pub. No. 2014/0170753, the disclosure of which hereby is incorporated by reference in its entirety.

### Zinc Finger Nuclease (ZFN)

In some embodiments, the B2M-modified NK-92 cells comprising a B2M-targeted alteration are produced by knocking out B2M in NK-92 cells with a zinc finger nuclease (ZFN). ZFNs are fusion proteins that comprise a non-specific cleavage domain (N) of FokI endonuclease and a zinc finger protein (ZFP). A pairs of ZNFs are involved to recognize a specific locus in a target gene—one that recognizes the sequence upstream and the other that recognizes the sequence downstream of the site to be modified—and the nuclease portion of the ZFN cuts at the specific locus and causing the knock out of the target gene. Methods of using the ZFNs to reduce gene expression is well known, for example, as disclosed in U.S. Pat. No. 9,045,763, and also in Durai et al., “Zinc Finger Nucleases: Custom-Designed Molecular Scissors for Genome Engineering of Plant and Mamalian cells,” Nucleic Acid Research 33 (18):5978-5990 (2005), the disclosures of which are incorporated by reference in its entirety.

### Transcription Activator-Like Effector Nucleases (TALENS)

In some embodiments, the B2M-modified NK-92 cells comprising a B2M-targeted alteration are produced by knocking out B2M in NK-92 cells with transcription activator-like effector nucleases (TALENS). TALENs are similar to ZFNs in that they bind as a pair around a genomic site and direct the same non-specific nuclease, FoKI, to cleave the genome at a specific site, but instead of recognizing DNA triplets, each domain recognizes a single nucleotide. Methods of using the ZFNs to reduce gene expression are also well known, for example, as disclosed in U.S. Pat. No. 9,005,973, and also Christian et al. “Targeting DNA Double-Strand Breaks with TAL Effector Nucleases,” Genetics 186(2): 757-761 (2010), the disclosures of which are incorporated by reference in their entirety.

### Knocking Down Beta 2 Microglobulin in NK-92 Cells

In some embodiments, the B2M-modified NK-92 cells comprising a B2M-targeted alteration is produced by knocking down B2M with an interfering RNA. Interfering RNAs, when introduced in vivo, forms a RNA-inducing silencing complex (“RISC”) with other proteins and initiate a process known as RNA interference (RNAi). During the RNAi process, the RISC incorporates a single-stranded interfering RNA or one strand of a double stranded interfering RNA. The incorporated strand acts as a template for RISC to recognize complementary mRNA transcript. Once the complementary mRNA is identified, the protein components in RISC activate and cleave the mRNA, resulting in a knock-down of target gene expression. Non-limiting examples of interfering RNA molecules that be used to knock down expression of B2M include siRNAs, short hairpin RNAs (shRNAs), single stranded interfering RNAs, and microRNAs (miRNAs). Methods for using these interfering RNAs are well known to one of skilled in the art.

In one embodiment, the interfering RNA is a siRNA. siRNA is a double stranded RNA which is typically less than 30 nucleotides long. Gene silencing by siRNA starts with one strand of the siRNA being incorporated into a ribonucleoprotein complex known as the RNA-induced silencing complex (RISC). The strand incorporated in RISC identifies mRNA molecules that are at least partially complementary to the incorporated siRNA strand and the RISC then cleaves these target mRNAs or inhibits their translation.

In one embodiment, the interfering RNA is a microRNA. microRNA is a small non-coding RNA molecule, which can hybridize to complementary sequences within mRNA molecules, resulting cleavage of the mRNA, or destabilization of the mRNA through shortening of its poly(A) tail.

In one embodiment, the interfering RNA is a single-stranded interfering RNA. The single strand can also effect mRNA silencing in a manner that is similar to the double stranded siRNA, albeit less efficient than, the double-stranded siRNA. The single-stranded interfering RNA typically has a length of about 19 to about 49 nucleotides as for the double-stranded siRNA described above.

A short hairpin RNA or small hairpin RNA (shRNA) is an artificial RNA molecule with a tight hairpin turn that can be used to silence target gene expression via the siRNA it produced in cells. Expression of shRNA in cells is typically accomplished by delivery of plasmids or through viral or bacterial vectors. Suitable bacterial vectors include but not limited to adeno-associated viruses (AAVs), adenoviruses, and lentiviruses. shRNA is an advantageous mediator of siRNA in that it has relatively low rate of degradation and turnover.

Interfering RNAs used in the invention may differ from naturally-occurring RNA by the addition, deletion, substitution or modification of one or more nucleotides. Non-nucleotide material may be bound to the interfering RNA, either at the 5′ end, the 3′ end, or internally. Non-limiting examples of modifications that interfering RNAs may contain relative to the naturally-occurring RNA are disclosed in U.S. Pat. No. 8,399,653, herein incorporated by reference in its entirety. Such modifications are commonly designed to increase the nuclease resistance of the interfering RNAs, to improve cellular uptake, to enhance cellular targeting, to assist in tracing the interfering RNA, to further improve stability, or to reduce the potential for activation of the interferon pathway. For example, interfering RNAs may comprise a purine nucleotide at the ends of overhangs. Conjugation of cholesterol to the 3′ end of the sense strand of an siRNA molecule by means of a pyrrolidine linker, for example, also provides stability to an siRNA.

Interfering RNAs used in the invention are typically about 10-60, 10-50, or 10-40 (duplex) nucleotides in length, more typically about 8-15, 10-30, 10-25, or 10-25 (duplex) nucleotides in length, about 10-24, (duplex) nucleotides in length (e.g., each complementary sequence of the double-stranded siRNA is 10-60, 10-50, 10-40, 10-30, 10-25, or 10-25 nucleotides in length, about 10-24, 11-22, or 11-23 nucleotides in length, and the double-stranded siRNA is about 10-60, 10-50, 10-40, 10-30, 10-25, or 10-25 base pairs in length).

Techniques for selecting target motifs in a gene of interest for RNAi are known to those skilled in the art, for example, as disclosed in Tuschl, T. et al., “The siRNA User Guide,” revised May 6, 2004, available on the Rockefeller University web site; by Technical Bulletin #506, “siRNA Design Guidelines,” Ambion Inc. at Ambion's web site; and by other web-based design tools at, for example, the Invitrogen, Dharmacon, Integrated DNA Technologies, Genscript, or Proligo web sites. Initial search parameters can include G/C contents between 35% and 55% and siRNA lengths between 19 and 27 nucleotides. The target sequence may be located in the coding region or in the 5′ or 3′ untranslated regions of the mRNA. The target sequences can be used to derive interfering RNA molecules, such as those described herein.

Efficiency of the knock-out or knock-down can be assessed by measuring the amount of B2M mRNA or protein using methods well known in the art, for example, quantitative PCR, western blot, flow cytometry, etc and the like. In some embodiments, the level of B2M protein is evaluated to assess knock-out or knock-down efficiency. In certain embodiments, the efficiency of reduction of B2M expression is at least 5%, at least 10%, at least 20%, at least 30%, at least 50%, at least 60%, or at least 80% as compared to B2M-unmodified NK-92 cells. In certain embodiments, the efficiency of reduction is from about 10% to about 90%. In certain embodiments, the efficiency of reduction is from about 30% to about 80%. In certain embodiments, the efficiency of reduction is from about 50% to about 80%. In some embodiments, the efficiency of reduction is greater than or equal to about 80%.

### HLA-E Modifications

In some embodiments, the disclosure provides B2M-modified NK-92 cells that also express HLA-E on the cell surface. Patients' endogenous NK cells will recognize HLA-E through receptor CD94/NKG2A or CD94/NKG2B. Not to be bound by theory, the interaction between the receptor and HLA-E results in inhibition of the cytotoxic activity of endogenous NK cells. Accordingly, the present invention provides for B2M-modified NK-92 cells having a B2M targeted alteration, and any one or more of the further modifications described above, are further modified to express a single chain trimer comprising an HLA-E leader peptide (which is normally bound by HLA-E), the mature form of B2M, and the mature HLA-E heavy chain. In some embodiments, the trimer comprises linker sequences between the coding sequences of the HLA-binding peptide, B2M, and the HLA-E heavy chain. HLA-E binding peptides are from the leader sequences of other HLA class I molecules, e.g., HLA-A, HLA-B, or HLA-C. For example, in one embodiment, the HLA-E binding peptide is the leader sequence of HLA-A*0201 and has a sequence of VMAPRTLVL (SEQ ID NO:20). In one embodiment, the HLA-E heavy chain polypeptide comprised by the trimer peptide comprises the amino acid sequence corresponding to the mature polypeptide region of SEQ ID NO:7, i.e., comprises amino acids 22-358 of SEQ ID NO:7. In an alternative embodiment, the HLA-E heavy chain polypeptide comprised by the trimer peptide comprises the amino acid sequence of SEQ ID NO:16.

A trimeric single chain HLA-E molecule has been successfully used in xenotransplantation experiments to protect porcine endothelial cells from killing by human NK cells (Crew et al Mol Immunol 2005 and Lilienfelde et al Xenotransplantation 2007). In these studies, the peptide employed corresponds to the leader peptide of human HLA-Cw*0304. As noted above, leader peptides from other HLA class I molecules can also be used, since they have been shown to bind HLA-E and inhibit killing mediated by CD94/NKG2A+ NK cell clones (see, e.g., Braud et al Nature 1998 and Eur. J. Immunol. 1997).

As described above, in some embodiments, the trimer can comprise linker sequences. In some embodiments, the linker is a flexible linker, e.g., containing amino acids such as Gly, Asn, Ser, Thr, Ala, and the like. Such linkers are designed using known parameters. For example, the linker may have repeats, such as Gly-Ser repeats.

### Additional Modifications

### Fc Receptors

In some embodiments the B2M-modified NK-92 cells comprising the B2M-targeted alteration are further modified to express a Fc receptor on the cell surface. For example, in some embodiments, e.g., in which B2M-modified NK-92 cells are administered with a monoclonal antibody, the Fc receptor allows the NK cells to work in unison with antibodies that kill target cells through ADCC. In some embodiments, the Fc receptor is IgG Fc receptor FcγRIII. In some embodiments, the Fc receptor is the high affinity form of the transmembrane immunoglobulin γ Fc region receptor III-A (CD16) in which a valine is present at position 158 of the mature form of the polypeptide).

Non-limiting examples of Fc receptors are provided below. These Fc receptors differ in their preferred ligand, affinity, expression, and effect following binding to the antibody.

TABLE 1
Illustrative Fc receptors

| | Principal | Affinity | | |
|---|---|---|---|---|
| Receptor | antibody | for | | Effect following binding |
| name | ligand | ligand | Cell distribution | to antibody |
| FcγRI (CD64) | IgG1 and | High | Macrophages | Phagocytosis |
| | IgG3 | (Kd~ | Neutrophils | Cell activation |
| | | 10⁻⁹ M) | Eosinophils | Activation of respiratory |
| | | | Dendritic cells | burst |
| | | | | Induction of microbe |
| | | | | killing |
| FcγRIIA (CD32) | IgG | Low | Macrophages | Phagocytosis |
| | | (Kd > | Neutrophils | Degranulation (eosinophils) |
| | | 10⁻⁷ M) | Eosinophils | |
| | | | Platelets | |
| | | | Langerhans cells | |
| FcγRIIB1 (CD32) | IgG | Low | B Cells | No phagocytosis |
| | | (Kd > | Mast cells | Inhibition of cell activity |
| | | 10⁻⁷ M) | | |
| FcγRIIB2 (CD32) | IgG | Low | Macrophages | Phagocytosis |
| | | (Kd > | Neutrophils | Inhibition of cell activity |
| | | 10⁻⁷ M) | Eosinophils | |
| FcγRIIIA (CD16a) | IgG | Low | NK cells | Induction of antibody- |
| | | (Kd > | Macrophages (certain | dependent cell-mediated |
| | | 10⁻⁶ M) | tissues) | cytotoxicity (ADCC) |
| | | | | Induction of cytokine |
| | | | | release by macrophages |
| FcγRIIIB (CD16b) | IgG | Low | Eosinophils | Induction of microbe |
| | | (Kd > | Macrophages | killing |
| | | 10⁻⁶ M) | Neutrophils | |
| | | | Mast cells | |
| | | | Follicular dendritic | |
| | | | cells | |
| FcεRI | IgE | High | Mast cells | Degranulation |
| | | (Kd~ | Eosinophils | Phagocytosis |
| | | 10⁻¹⁰ M) | Basophils | |
| | | | Langerhans cells | |
| | | | Monocytes | |
| FcεRII (CD23) | IgE | Low | B cells | Possible adhesion molecule |
| | | (Kd > | Eosinophils | IgE transport across human |
| | | 10⁻⁷ M) | Langerhans cells | intestinal epithelium |
| | | | | Positive-feedback |
| | | | | mechanism to enhance |
| | | | | allergic sensitization (B |
| | | | | cells) |
| FcαRI (CD89) | IgA | Low | Monocytes | Phagocytosis |
| | | (Kd > | Macrophages | Induction of microbe |
| | | 10⁻⁶ M) | Neutrophils | killing |
| | | | Eosinophils | |
| Fcα/μR | IgA and IgM | High for | B cells | Endocytosis |
| | | IgM, | Mesangial cells | Induction of microbe |
| | | Mid for | Macrophages | killing |
| | | IgA | | |
| FcRn | IgG | | Monocytes | Transfers IgG from a |
| | | | Macrophages | mother to fetus through the |
| | | | Dendritic cells | placenta |
| | | | Epithelial cells | Transfers IgG from a |
| | | | Endothelial cells | mother to infant in milk |
| | | | Hepatocytes | Protects IgG from |
| | | | | degradation |

In some embodiments, the Fc receptor is CD16. In typical embodiments, NK-92 cells are modified to express a high affinity form of human CD16 having a valine at position 158 of the mature form of the protein, e.g., SEQ ID NO:5. Position 158 of the mature protein corresponds to position 176 of the human CD16 sequence that includes the native signal peptide.

In some embodiments, the CD16 has at least 70%, at least 80%, at least 90%, or at least 95% identity to SEQ ID NO:5 and comprises a valine at position 158 as determined with reference to SEQ ID NO:5.

### Chimeric Antigen Receptors

In some embodiments, the B2M-modified NK-92 cells are further engineered to express a chimeric antigen receptor (CAR) on the cell surface. Optionally, the CAR is specific for a tumor-specific antigen. Tumor-specific antigens are described, by way of non-limiting example, in US 2013/0189268; WO 1999024566 A1; U.S. Pat. No. 7,098,008; and WO 2000020460 A1, each of which is incorporated herein by reference in its entirety. Tumor-specific antigens include, without limitation, NKG2D, CS1, GD2, CD138, EpCAM, EBNA3C, GPA7, CD244, CA-125, ETA, MAGE, CAGE, BAGE, HAGE, LAGE, PAGE, NY-SEO-1, GAGE, CEA, CD52, CD30, MUCSAC, c-Met, EGFR, FAB, WT-1, PSMA, NY-ESO1, AFP, CEA, CTAG1B, CD19 and CD33. Additional non-limiting tumor-associated antigens, and the malignancies associated therewith, can be found in Table 2.

TABLE 2
Tumor-Specific Antigens and Associated Malignancies

| Target Antigen | Associated Malignancy |
|---|---|
| α-Folate | Ovarian Cancer |
| Receptor | |
| CAIX | Renal Cell Carcinoma |
| CD19 | B-cell Malignancies |
| | Chronic lymphocytic leukemia (CLL) |
| | B-cell CLL (B-CLL) |
| | Acute lymphoblastic leukemia (ALL); ALL |
| | post Hematopoietic stem cell transplantation |
| | (HSCT) |
| | Lymphoma; Refractory Follicular |
| | Lymphoma; B-cell non-Hodgkin lymphoma |
| | (B-NHL) |
| | Leukemia |
| | B-cell Malignancies post-HSCT |
| | B-lineage Lymphoid Malignancies post |
| | umbilical cord blood transplantation (UCBT) |
| CD19/CD20 | Lymphoblastic Leukemia |
| CD20 | Lymphomas |
| | B-Cell Malignancies |
| | B-cell Lymphomas |
| | Mantle Cell Lymphoma |
| | Indolent B-NHL |
| | Leukemia |
| CD22 | B-cell Malignancies |
| CD30 | Lymphomas; Hodgkin Lymphoma |
| CD33 | AML |
| CD44v7/8 | Cervical Carcinoma |
| CD138 | Multiple Myeloma |
| CD244 | Neuroblastoma |
| CEA | Breast Cancer |
| | Colorectal Cancer |
| CS1 | Multiple Myeloma |
| EBNA3C | EBV Positive T-cells |
| EGP-2 | Multiple Malignancies |
| EGP-40 | Colorectal Cancer |
| EpCAM | Breast Carcinoma |
| Erb-B2 | Colorectal Cancer |
| | Breast Cancer and Others |
| | Prostate Cancer |
| Erb-B 2,3,4 | Breast Cancer and Others |
| FBP | Ovarian Cancer |
| Fetal | Rhabdomyosarcoma |
| Acetylcholine Receptor | |
| GD2 | Neuroblastoma |
| GD3 | Melanoma |
| GPA7 | Melanoma |
| Her2 | Breast Carcinoma |
| | Ovarian Cancer |
| | Tumors of Epithelial Origin |
| Her2/new | Medulloblastoma |
| | Lung Malignancy |
| | Advanced Osteosarcoma |
| | Glioblastoma |
| IL-13R-a2 | Glioma |
| | Glioblastoma |
| | Medulloblastoma |
| KDR | Tumor Neovasculature |
| k-light chain | B-cell Malignancies |
| | B-NHL, CLL |
| LeY | Carcinomas |
| | Epithelial Derived Tumors |
| L1 Cell | Neuroblastoma |
| Adhesion Molecule | |
| MAGE-A1 | Melanoma |
| Mesothelin | Various Tumors |
| MUC1 | Breast Cancer; Ovarian Cancer |
| NKG2D Ligands | Various Tumors |
| Oncofetal | Various Tumors |
| Antigen (h5T4) | |
| PSCA | Prostate Carcinoma |
| PSMA | Prostate/Tumor Vasculature |
| TAA Targeted | Various Tumors |
| by mAb IgE | |
| TAG-72 | Adenocarcinomas |
| VEGF-R2 | Tumor Neovasculature |

In some embodiments, the CAR targets CD19, CD33 or CSPG-4. In some embodiments, the CAR targets an antigen associated with a specific cancer type. For example, the cancer may be selected from the group consisting of leukemia (including acute leukemias (e.g., acute lymphocytic leukemia, acute myelocytic leukemia (including myeloblastic, promyelocytic, myelomonocytic, monocytic, and erythroleukemia)) and chronic leukemias (e.g., chronic myelocytic (granulocytic) leukemia and chronic lymphocytic leukemia), polycythemia vera, lymphomas (e.g., Hodgkin's disease and non-Hodgkin's disease), multiple myeloma, Waldenstrom's macroglobulinemia, heavy chain disease, solid tumors including, but not limited to, sarcomas and carcinomas such as fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, menangioma, melanoma, neuroblastoma and retinoblastoma.

CARs can be engineered as described, for example, in Patent Publication Nos. WO 2014039523; US 20140242701; US 20140274909; US 20130280285; and WO 2014099671, each of which is incorporated herein by reference in its entirety. Optionally, the CAR is a CD19 CAR, a CD33 CAR or CSPG-4 CAR.

### Cytokines

In some embodiments, the invention provides B2M-modified NK-92 cells that a further modified to express at least one cytokine. In such cells, the expression of cytokines in the cells is typically directed to the endoplasmic reticulum. This feature prevents undesirable effects of systemic administration of cytokines, such as toxicity affecting the cardiovascular, gastrointestinal, respiratory and nervous systems. In some embodiments, the at least one cytokine is IL-2, IL-12, IL-15, IL-18, IL-21 or a variant thereof. In preferred embodiments, the cytokine is IL-2, e.g., human IL-2.

In certain embodiments the IL-2 is a variant that is targeted to the endoplasmic reticulum. Thus, for example, the IL-2 is expressed with a signal sequence that directs the IL-2 to the endoplasmic reticulum. In some embodiments, the IL-2 is human IL-2. Not to be bound by theory, but directing the IL-2 to the endoplasmic reticulum permits expression of IL-2 at levels sufficient for autocrine activation, but without releasing IL-2 extracellularly. See Konstantinidis et al “Targeting IL-2 to the endoplasmic reticulum confines autocrine growth stimulation to NK-92 cells” Exp Hematol. 2005 February; 33(2):159-64.

In some embodiments, a suicide gene may also be inserted into B2M-modified NK-92 cells, e.g., in B2M-modified NK-92 cells that express IL-2 to prevent unregulated endogenous expression of IL-2, that could lead to the potential development of mutants with autonomous growth. In some embodiments, the suicide gene is icaspase 9 (iCas9).

### Transgene Expression

Also encompassed in the disclosure are sequences that share significant sequence identity to the polynucleotides or polypeptides described above, e.g., Cas proteins, HLA-E, CD16, Fc receptor, CAR, and/or IL-2. These sequences can also be introduced into the B2M-unmodified NK-92 cells. In some embodiments, the sequences have at least 70%, at least 80%, at least 85%, at least 88%, at least 95%, or at least 98%, or at least 99% sequence identity to their respective native sequences.

Transgenes (e.g. Cas proteins, HLA-E, CD16, Fc receptor, CAR, and/or IL-2) can be engineered into an expression plasmid by any mechanism known to those of skill in the art. Transgenes may be engineered into the same expression plasmid or different. In preferred embodiments, the transgenes are expressed on the same plasmid.

Transgenes can be introduced into NK-92 cells using any transient transfection method known in the art, including, for example, electroporation, lipofection, nucleofection, or “gene-gun.”

Any number of vectors can be used to express these transgenes. In some embodiments, the vector is a retroviral vector. In some embodiments, the vector is a plasmid vector. Other viral vectors that can be used include adenoviral vectors, adeno-associated viral vectors, herpes simplex viral vectors, pox viral vectors, and others.

### Combination Therapies

In some embodiments, B2M-modified NK-92 cells of the present disclosure are used in combination with therapeutic antibodies and/or other anti-cancer agents. Therapeutic antibodies may be used to target cells that are infected or express cancer-associated markers. Examples of cancer therapeutic monoclonal antibodies are shown in Table 3.

TABLE 3
Illustrative therapeutic monoclonal antibodies
Examples of FDA-approved therapeutic monoclonal antibodies

| | Brand | | | Indication |
|---|---|---|---|---|
| Antibody | name | Company | Target | (Targeted disease) |
| Alemtuzumab | Campath ® | Genzyme | CD52 | Chronic lymphocytic leukemia |
| Brentuximab | Adcetris ® | | CD30 | Anaplastic large cell |
| vedotin | | | | lymphoma (ALCL) and Hodgkin |
| | | | | lymphoma |
| Cetuximab | Erbitux ® | Bristol-Myers | epidermal growth | Colorectal cancer, Head and |
| | | Squibb/Eli | factor receptor | neck cancer |
| | | Lilly/Merck | | |
| | | KGaA | | |
| Gemtuzumab | Mylotarg ® | Wyeth | CD33 | Acute myelogenous |
| | | | | leukemia (with calicheamicin) |
| Ibritumomab | Zevalin ® | Spectrum | CD20 | Non-Hodgkin |
| tiuxetan | | Pharmaceuticals, | | lymphoma (with yttrium- |
| | | Inc. | | 90 or indium-111) |
| Ipilimumab | Yervoy ® | | blocks CTLA-4 | Melanoma |
| (MDX-101) | | | | |
| Ofatumumab | Arzerra ® | | CD20 | Chronic lymphocytic leukemia |
| Palivizumab | Synagis ® | MedImmune | an epitope of the RSV | Respiratory Syncytial Virus |
| | | | F protein | |
| Panitumumab | Vectibix ® | Amgen | epidermal growth | Colorectal cancer |
| | | | factor receptor | |
| Rituximab | Rituxan ®, | Biogen | CD20 | Non-Hodgkin lymphoma |
| | Mabthera ® | Idec/Genentech | | |
| Tositumomab | Bexxar ® | GlaxoSmithKline | CD20 | Non-Hodgkin lymphoma |
| Trastuzumab | Herceptin ® | Genentech | ErbB2 | Breast cancer |
| Blinatunomab | | | bispecific CD19- | Philadelphia chromosome- |
| | | | directed CD3 T-cell | negative relapsed or |
| | | | engager | refractory B cell precursor |
| | | | | acute lymphoblastic leukemia |
| | | | | (ALL) |
| Avelumamab | | | anti-PD-L1 | Non-small cell lung cancer, |
| | | | | metastatic Merkel cell |
| | | | | carcinoma; gastic cancer, |
| | | | | breast cancer, ovarian cancer, |
| | | | | bladder cancer, melanoma, |
| | | | | meothelioma, including |
| | | | | metastatic or locally advanced |
| | | | | solid tumors |
| Daratumumab | | | CD38 | Multiple myeloma |
| Elotuzumab | | | a SLAMF7-directed | Multiple myeloma |
| | | | (also known as CD | |
| | | | 319) | |
| | | | immunostimulatory | |
| | | | antibody | |

Antibodies may treat cancer through a number of mechanisms. Antibody-dependent cellular cytotoxicity (ADCC) occurs when immune cells, such as B2M-modified NK cells of the present disclosure that also expresses FcR, bind to antibodies that are bound to target cells through Fc receptors, such as CD16. Accordingly, in some embodiments, B2M-modified NK-92 cells expressing FcR are administered to a patient along with antibodies directed against a specific cancer-associated protein. Administration of such NK-92 cells may be carried out simultaneously with the administration of the monoclonal antibody, or in a sequential manner. In some embodiments, the NK-92 cells are administered to the subject after the subject has been treated with the monoclonal antibody. Alternatively, the B2M-modified NK-92 cells maybe administered at the same time, e.g., within 24 hours, of the monoclonal antibody.

In some embodiments, B2M-modified NK-92 cells are administered intravenously. In some embodiments the FcR-expressing NK-92 cells are infused directly into the bone marrow.

### Treatment

Also provided are methods of treating patients with B2M-NK-92 cells as described herein. In some embodiments, the patient is suffering from cancer or an infectious disease. As described above, B2M-NK-92 cells may be further modified to express a CAR that targets an antigen expressed on the surface of the patient's cancer cells. In some embodiments, B2M-modified NK-92 cells may also expressed and Fc receptor, e.g., CD16. In some embodiments, the patient is treated with B2M-modified NK-92 cell and also an antibody. B2M-modified NK-92 cells can be administered to an individual by absolute numbers of cells, e.g., said individual can be administered from about 1000 cells/injection to up to about 10 billion cells/injection, such as at about, at least about, or at most about, 1×10⁸, 1×10⁷, 5×10⁷, 1×10⁶, 5×10⁶, 1×10⁵, 5×10⁵, 1×10⁴, 5×10⁴, 1×10³, 5×10³ (and so forth)NK-92 cells per injection, or any ranges between any two of the numbers, end points inclusive.

In other embodiments, said individual can be administered from about 1000 cells/injection/m² to up to about 10 billion cells/injection/m², such as at about, at least about, or at most about, 1×10⁸/m², 1×10⁷/m², 5×10⁷/m², 1×10⁶/m², 5×10⁶/m², 1×10⁵/m², 5×10⁵/m², 1×10⁴/m², 5×10⁴/m², 1×10³/m², 5×10³/m² (and so forth) NK-92 cells per injection, or any ranges between any two of the numbers, end points inclusive.

In other embodiments, B2M-modified NK-92 cells can be administered to such individual by relative numbers of cells, e.g., said individual can be administered about 1000 cells to up to about 10 billion cells per kilogram of the individual, such as at about, at least about, or at most about, 1×10⁸, 1×10⁷, 5×10⁷, 1×10⁶, 5×10⁶, 1×10⁵, 5×10⁵, 1×10⁴, 5×10⁴, 1×10³, 5×10³ (and so forth) NK-92 cells per kilogram of the individual, or any ranges between any two of the numbers, end points inclusive.

In other embodiments, the total dose may be calculated by m² of body surface area, including about 1×10¹¹, 1×10¹⁰, 1×10⁹, 1×10⁸, 1×10⁷, per m², or any ranges between any two of the numbers, end points inclusive. The average person is about 1.6 to about 1.8 m². In a preferred embodiment, between about 1 billion and about 3 billion NK-92 cells are administered to a patient. In other embodiments, the amount of NK-92 cells injected per dose may calculated by m² of body surface area, including 1×10¹¹, 1×10¹⁰, 1×10⁹, 1×10⁸, 1×10⁷, per m². The average person is 1.6-1.8 m².

B2M-modified NK-92 cells, and optionally other anti-cancer agents can be administered once to a patient with cancer can be administered multiple times, e.g., once every 1, 2, 3, 4, 5, 6, 7, 8, 9, 10, 11, 12, 13, 14, 15, 16, 17, 18, 19, 20, 21, 22 or 23 hours, or once every 1, 2, 3, 4, 5, 6 or 7 days, or once every 1, 2, 3, 4, 5, 6, 7, 8, 9, 10 or more weeks during therapy, or any ranges between any two of the numbers, end points inclusive.

In some embodiments, B2M-modified NK-92 cells are administered in a composition comprising the B2M-modified NK-92 cells and a medium, such as human serum or an equivalent thereof. In some embodiments, the medium comprises human serum albumin. In some embodiments, the medium comprises human plasma. In some embodiments, the medium comprises about 1% to about 15% human serum or human serum equivalent. In some embodiments, the medium comprises about 1% to about 10% human serum or human serum equivalent. In some embodiments, the medium comprises about 1% to about 5% human serum or human serum equivalent. In a preferred embodiment, the medium comprises about 2.5% human serum or human serum equivalent. In some embodiments, the serum is human AB serum. In some embodiments, a serum substitute that is acceptable for use in human therapeutics is used instead of human serum. Such serum substitutes may be known in the art, or developed in the future. Although concentrations of human serum over 15% can be used, it is contemplated that concentrations greater than about 5% will be cost-prohibitive. In some embodiments, NK-92 cells are administered in a composition comprising NK-92 cells and an isotonic liquid solution that supports cell viability. In some embodiments, NK-92 cells are administered in a composition that has been reconstituted from a cryopreserved sample.

Pharmaceutically acceptable compositions can include a variety of carriers and excipients. A variety of aqueous carriers can be used, e.g., buffered saline and the like. These solutions are sterile and generally free of undesirable matter. Suitable carriers and excipients and their formulations are described in Remington: The Science and Practice of Pharmacy, 21st Edition, David B. Troy, ed., Lippicott Williams & Wilkins (2005). By pharmaceutically acceptable carrier is meant a material that is not biologically or otherwise undesirable, i.e., the material is administered to a subject without causing undesirable biological effects or interacting in a deleterious manner with the other components of the pharmaceutical composition in which it is contained. If administered to a subject, the carrier is optionally selected to minimize degradation of the active ingredient and to minimize adverse side effects in the subject. As used herein, the term pharmaceutically acceptable is used synonymously with physiologically acceptable and pharmacologically acceptable. A pharmaceutical composition will generally comprise agents for buffering and preservation in storage and can include buffers and carriers for appropriate delivery, depending on the route of administration.

These compositions for use in in vivo or in vitro may be sterilized by sterilization techniques employed for cells. The compositions may contain acceptable auxiliary substances as required to approximate physiological conditions such as pH adjusting and buffering agents, toxicity adjusting agents and the like, for example, sodium acetate, sodium chloride, potassium chloride, calcium chloride, sodium lactate and the like. The concentration of cells in these formulations and/or other agents can vary and will be selected primarily based on fluid volumes, viscosities, body weight and the like in accordance with the particular mode of administration selected and the subject's needs.

In one embodiment, B2M-modified NK-92 cells are administered to the patient in conjunction with one or more other treatments for the cancer being treated. In some embodiments, two or more other treatments for the cancer being treated includes, for example, an antibody, radiation, chemotherapeutic, stem cell transplantation, or hormone therapy.

In one embodiment, B2M-modified NK-92 cells are administered in conjunction with an antibody targeting the diseased cells. In one embodiment, B2M-modified NK-92 cells and an antibody are administered to the patient together, e.g., in the same formulation; separately, e.g., in separate formulations, concurrently; or can be administered separately, e.g., on different dosing schedules or at different times of the day. When administered separately, the antibody can be administered in any suitable route, such as intravenous or oral administration.

In some embodiments, B2M-modified NK-92 cells that also express an FcR, e.g., a high affinity CD16 that expresses FcR, may be carried out simultaneously with administration of a monoclonal antibody, or in a sequential manner. In some embodiments, the FcR-expressing NK-92 cells are administered to the subject within 24 hours after the subject has been treated with the monoclonal antibody.

### Kits

Also disclosed are kits for the treatment of cancer or an infectious disease using compositions comprising an amount of B2M-modified NK-92 cells as described herein. In some embodiments, the kits of the present disclosure may also include at least one monoclonal antibody.

In certain embodiments, the kit may contain additional compounds such as therapeutically active compounds or drugs that are to be administered before, at the same time or after administration of B2M-modified NK-92 cells. Examples of such compounds include an antibody, vitamins, minerals, fludrocortisone, ibuprofen, lidocaine, quinidine, chemotherapeutic, etc.

In various embodiments, instructions for use of the kits will include directions to use the kit components in the treatment of a cancer or an infectious disease. The instructions may further contain information regarding how to B2M-modified NK-92 cells (e.g., thawing and/or culturing). The instructions may further include guidance regarding the dosage and frequency of administration.

Disclosed are materials, compositions, and components that can be used for, can be used in conjunction with, can be used in preparation for, or are products of the disclosed methods and compositions. These and other materials are disclosed herein, and it is understood that when combinations, subsets, interactions, groups, etc. of these materials are disclosed that while specific reference of each various individual and collective combinations and permutations of these compounds may not be explicitly disclosed, each is specifically contemplated and described herein. For example, if a method is disclosed and discussed and a number of modifications that can be made to a number of molecules including the method are discussed, each and every combination and permutation of the method, and the modifications that are possible are specifically contemplated unless specifically indicated to the contrary. Likewise, any subset or combination of these is also specifically contemplated and disclosed. This concept applies to all aspects of this disclosure including, but not limited to, steps in methods using the disclosed compositions. Thus, if there are a variety of additional steps that can be performed, it is understood that each of these additional steps can be performed with any specific method steps or combination of method steps of the disclosed methods, and that each such combination or subset of combinations is specifically contemplated and should be considered disclosed.

### EXAMPLES

The following examples are for illustrative purposes only and should not be interpreted as limitations of the claimed invention. There are a variety of alternative techniques and procedures available to those of skill in the art which would similarly permit one to successfully perform the intended invention.

### Example 1: Analysis of Immunogenicity of NK-92 Cells

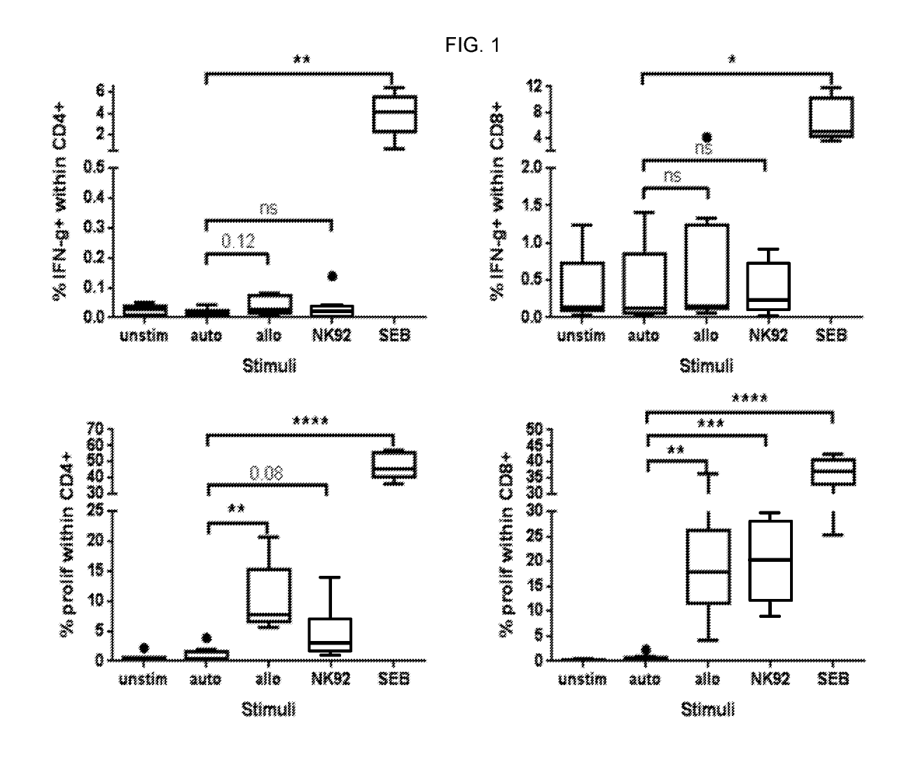

Initial evaluation of the immunogenicity of NK-92 cells was performed in Mixed Lymphocyte Reaction (MLR) experiments, in which PBMCs (peripheral blood mononuclear cells) from healthy donors were mixed with irradiated allogeneic PBMCs or NK-92 cells. As shown in FIG. 1, a proliferative response of CD8+ T cells was observed against allogeneic PBMCs and NK-92 cells. Staphylococcal enterotoxin B (SEB) superantigen was used as positive control for proliferation.

### Example 2: Generation of CAS9-NK-92 and CAS9-Hank Cell Lines

Cell lines stably expressing Cas9 protein were generated by infecting NK-92 and haNK parental cells with the Edit-R Cas9 lentivirus. In brief, Edit-R Cas9 lentivirus stocks were produced by transfecting 7×10⁶ 293 T cells per 10 cm petri dish with the following amount of plasmids: 7.5 μg Edit-R-Cas9 (Dharmacon, catalog # CAS10138), 5 μg pCMV-AR8.2, and 2.5 μg pCMV-VSV.G. The transfections were performed using Lipofectamine 3000 (Life Technologies, catalog # L3000-008) following manufacturer's instructions. Virus supernatants were collected 48 h post-transfection, and concentrated 10 fold using PEG-it Virus Precipitation Solution from System Biosciences (catalog # LV810A-1). 5×10⁵ NK-92 or haNK parental cells (NK-92 cells expressing a high affinity CD16) were infected by spinoculation (840 g for 99 min at 35° C.) with 100 μl of concentrated virus in 1 ml of final medium in a 24 well plate, in the presence of TransDux (System Biosciences, catalog # LV850A-1). 48 hours post-transduction the Cas9-expressing cells were selected by growing the cells in the presence of 15 μg/ml of blasticidin (InvivoGen, catalog # ant-bl-1).

NK-92 cells are quite refractory to DNA transfection. Most methods of transfection, either liposome-based or electroporation, are inefficient and result in poor cell recovery. As opposed to DNA transfection, RNA transfection using electroporation is highly efficient and consistently results in cell viability of 90% or higher (data not shown). Despite its better performance, efficient transfection of large RNA molecules can be a challenge. Thus, for purposes of this experiment, NK-92 and haNK cells stably expressing Cas9 were generated as described above. Cell lysates were prepared in RIPA lysis buffer (50 mM Tris-HCl pH 7.4, 150 mM NaCl, 1 mM EDTA, 1% Triton X-100, 1% sodium deoxycholate, 0.1% SDS) supplemented with 1 mM PMSF, 1 μg/ml aprotinin, 1 μg/ml leupeptin. Protein concentration was measured by BCA Protein Assay (Pierce). Total protein (10 μg) was resolved on 10% SDS-PAGE, transferred to Nitrocellulose membranes (Life Technologies, catalog # D323002) using an iBlot2 apparatus (Life Technologies), and probed with primary antibodies against Cas9 (Cell Signaling, catalog #14697) or anti-a Tubulin (Santa Cruz Biotechnology, sc-23948), followed by incubation with horseradish peroxidase-(HRP) conjugated sheep anti-mouse or anti-rabbit Ig (Amersham). Signals were developed using SuperSignal West Femto (Pierce).

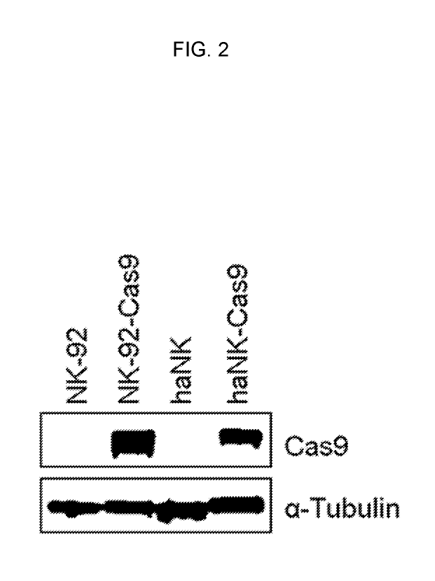

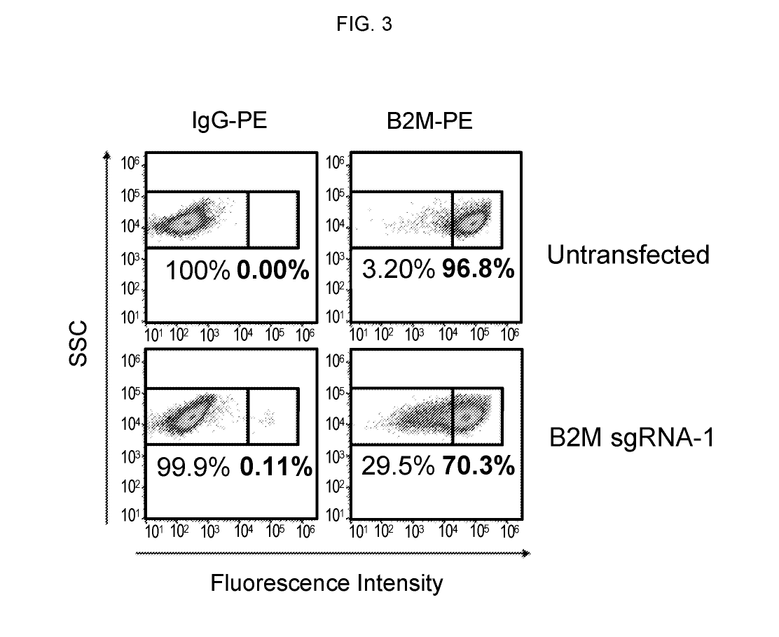

FIG. 2 shows that Cas9-NK-92 and Cas9-haNK cells express high levels of Cas9 protein. More importantly, these cell lines can be used for efficiently generating gene knock-outs. As shown in FIG. 3, transfection of B2M sgRNA-1 into NK-92 cells results in approx. 30% NK-92 cells negative for B2M expression. The flow cytometry analysis was performed 48 hours after transfection efficient KO in Cas9-NK-92 cells transfected with in vitro transcribed B2M sgRNA-1 RNA.

### Example 3: Generation of PT7-Guide-IVT B2M (Beta-2-Microglobulin) sgRNA Constructs

The guide RNAs were designed using the MIT web tool http://crispr.mit.edu. The sgRNAs target the first exon of human B2M, NM_004048.

| | | SEQ | | | | Location | Number of |
|---|---|---|---|---|---|---|---|
| Guide | Score | ID NO | Sequence (5′→3′) | PAM | Strand | in B2M ORF | off-target sites |
| #1 | 90 | 1 | GAGTAGCGCGAGCACAGCTA | AGG | minus | 20-39 | 38 (8 are in |
| | | | (SEQ ID NO: 1) | | | | genes) |
| #2 | 70 | 2 | CGCGAGCACAGCTAAGGCCA | CGG | minus | 14-33 | 129 (27 are |
| | | | (SEQ ID NO: 2) | | | | in genes) |
| #3 | 69 | 3 | CTCGCGCTACTCTCTCTTTC | TGG | plus | 28-47 | 121 (22 are |
| | | | (SEQ ID NO: 3) | | | | in genes) |
| #4 | 58 | 4 | GCTACTCTCTCTTTCTGGCC | TGG | plus | 33-52 | 258 (34 are |
| | | | (SEQ ID NO: 4) | | | | in genes) |

The B2M target sites were cloned into the pT7-Guide-IVT plasmid (Origene, catalog # GE100025). The oligos were cloned using the two BsmBI sites in pT7-Guide-IVT, and following manufacturer's instructions. In vitro transcribed B2M sgRNAs were generated using the MEGAshortscript™ T7 Kit (Life Technologies, catalog # AM1354), following the manufacturer's instructions.

### Example 4: Generation and Characterization of B2M-KO NK-92 Cells

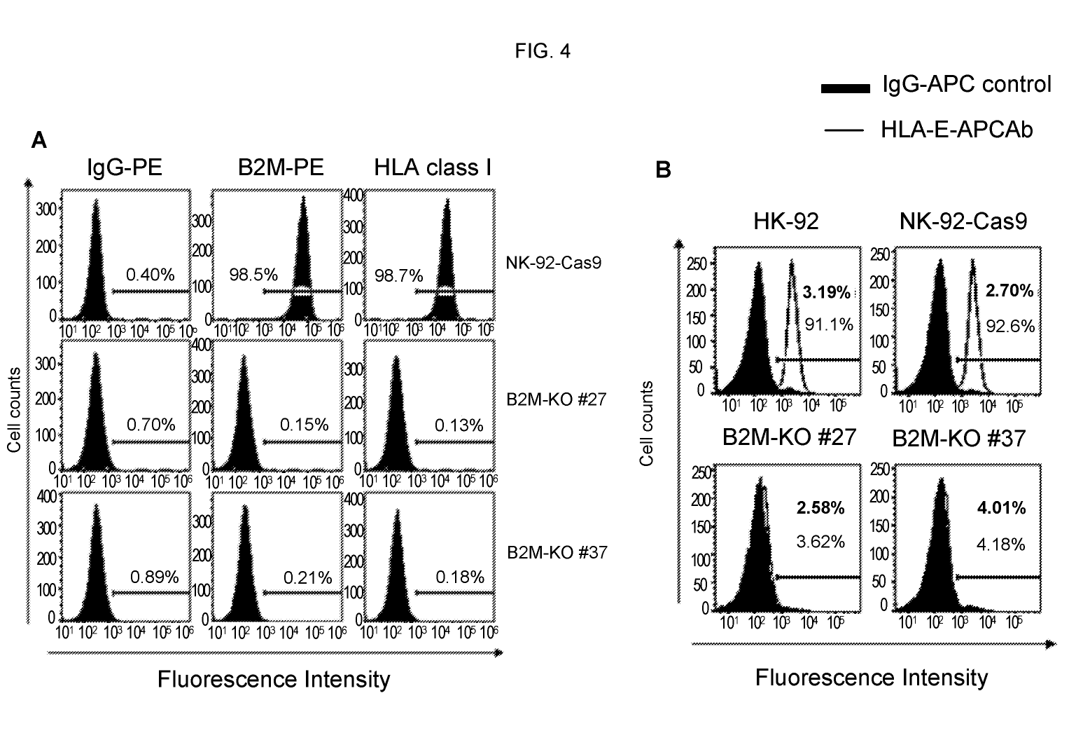

B2M-KO NK-92 cells were generated by transfecting Cas9-NK-92 cells with B2M sgRNA-1 RNA, using the MaxCyte GT electroporator. Briefly, 5×10⁶ Cas9-NK-92 cells were transfected with 10 μg of in vitro transcribed B2M sgRNA-1 RNA using NK-92-3-OC protocol. 48 hours post-transfection the cells were plated by limited dilution. After growing the cells for 15 days, individual clones were selected, expanded and tested for B2M expression by flow cytometry. FIG. 4 panels A and B show B2M and HLA class I expression of two representative B2M-KO NK-92 clones. As shown in FIG. 4, genetic ablation of the B2M gene in NK-92 cells leads to complete loss of HLA class I expression on the cell surface.

The anti-B2M-PE (cat #316306), anti-HLA-I-PE (cat #311406) and IgG1-PE control (cat #400114) antibodies were obtained from BioLegend. Cytofluorometric analyses were performed on a MACSQuant 10 flow cytometer (Miltenyi) and analyzed using FlowJo software.

### Example 5: HLA Class I-Deficient NK-92 Cells are Susceptible to Lysis by Allogeneic NK Cells

A potential pitfall of generating a less immunogenic HLA class I negative variant of NK-92, is that these cells might become susceptible to lysis by the recipient's NK cells. NK cell cytotoxic activity is determined by the balance between activating and inhibitory signals, mediated by multiple cell surface receptors. NK cells are known for monitoring HLA class I expression by use of cell surface receptors (KIRs and CD94/NKG2A) that transduce inhibitory signals and block NK cell-mediated lysis upon recognition of HLA class I molecules. Therefore, loss of HLA class I expression results in lack of receptor-mediated inhibition of NK cells, which may lead to their activation and lysis of the HLA class I-negative target.

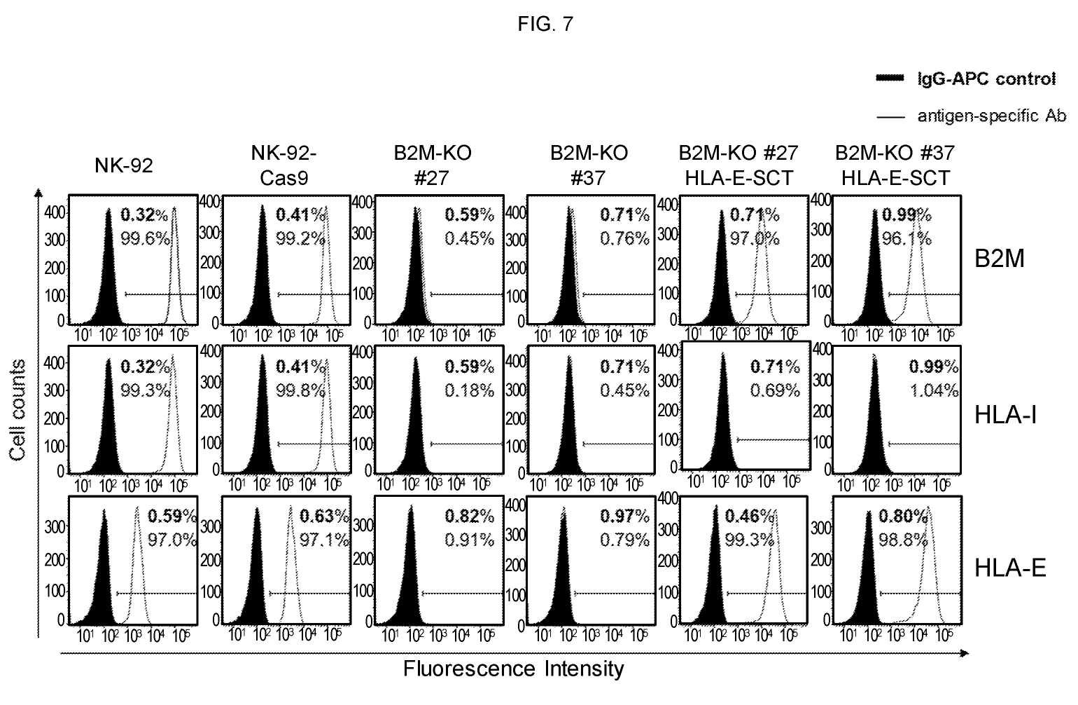

We evaluated the susceptibility of HLA-I deficient NK-92 cells to lysis by allogeneic NK cells in cytotoxicity experiments using either freshly purified (non-activated) or activated (IL-2 stimulated) NK cells from multiple donors as effectors. As shown in FIG. 7, NK-92 cells that do not express HLA class I molecules become highly susceptible to lysis by allogeneic NK cells, as compared to parental NK-92 cells. Their susceptibility is comparable to that of K562, an HLA-I deficient cell line highly susceptible to killing by NK cells.

### Example 6: Protection of HLA Class I-Deficient NK-92 Cells by Expression HLA-E as a Single Chain Trimer

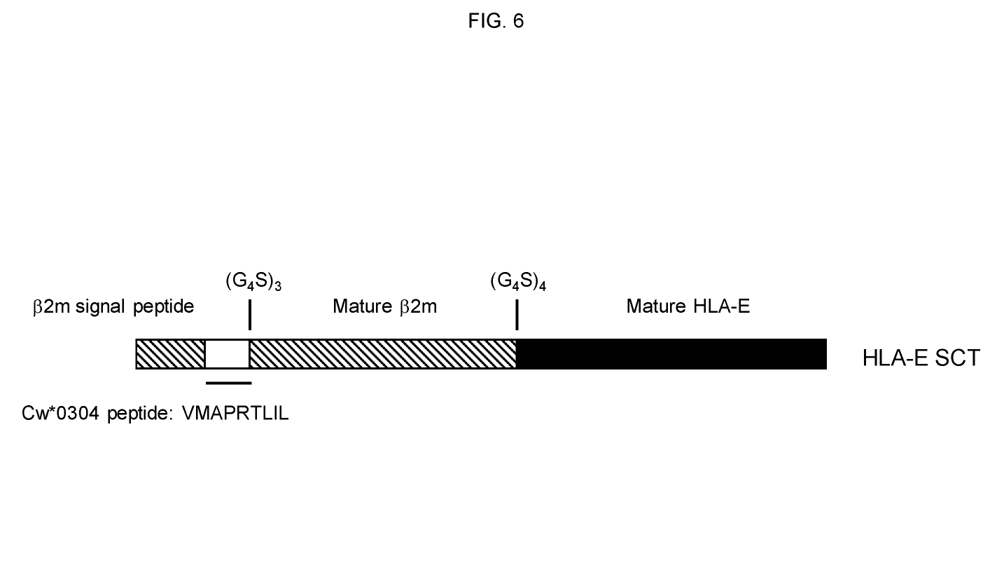

This example evaluates protective effects of expressing an HLA-E single chain trimer in HLA class I-deficient NK-92 cells. The design of an illustrative HLA-E single chang trimer is shown in FIG. 6.

### HLA-E-SCT Confers Partial Protection Against Allogeneic NK Cell Lysis

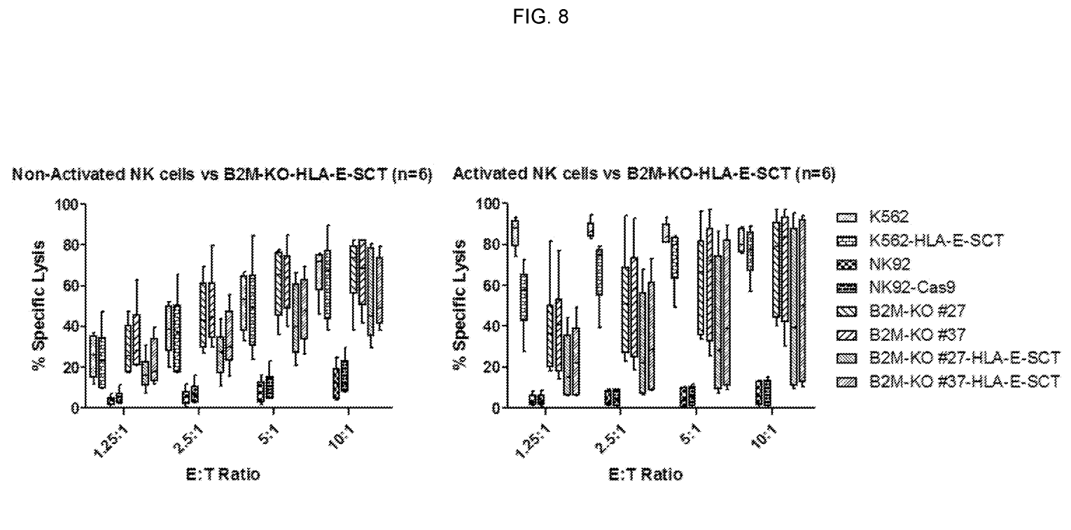

HLA-E binds peptides derived from the signal sequence of other classical HLA-I molecules, and is the ligand for the NK receptors CD94/NKG2A, CD94/NKG2B, and CD94/NKG2C. It has been shown that chimeric HLA-I molecules, consisting of an antigenic peptide, β2 microglobulin and HLA-I heavy chain expressed as a single molecule, can be efficiently displayed on the cell surface and recognized by their antigen receptor (Yu, et al., J Immunol 168:3145-9, 2002). In particular, enforced expression of HLA-E as a single chain trimer (SCT) has been used to prevent NK cell lysis of pig endothelial cells in xenotransplantation, and allogeneic pluripotent stem cells (PSCs) (Crew, et al., Mol Immunol 42:1205-14, 2005; Gornalusse, et al., Nat Biotechnol, 25:765-772, 2017). Expression of HLA-E as a single chain trimer was restored in HLA-I deficient NK-92 cells to evaluate whether expression protects HLA-I deficient NK-92 cells from allogeneic NK cell lysis. The chimeric HLA-E-SCT molecule encompasses the following elements: (32m signal peptide, Cw*0304 peptide (VMAPRTLIL, SEQ ID NO:12), (G₄S)₃ linker, mature β2m chain, (G₄S)₄ linker, and mature HLA-E chain (FIG. 6). Although this chimeric protein is based on that of Crew et al, supra, an important difference is that the designed used in this example corresponds to the HLA-E^{G} (E*0101 allele) allele, which contains a Gly at position 107, and has been shown to exhibit higher affinity for most peptides and higher thermal stability (Strong, et al., J Biol Chem: 278:5082-90, 2003). As shown in FIG. 7, enforced expression of HLA-E-SCT in two different HLA-I deficient NK-92 clones restored HLA-E expression to levels higher than those of parental cells. Importantly, since the β2m chain is covalently linked to the mature HLA-E chain the cells remain deficient for expression of classical HLA-A, -B, and -C molecules (FIG. 7). Despite high levels of expression of HLA-E in HLA-E-SCT expressing B2M-KO NK-92 cells, in the present example, HLA-E conferred partial protection against lysis by non-activated or activated allogeneic NK cells (FIG. 8). Not to be bound by theory, this is likely due to restricted expression of the inhibitory CD94/NKG2A receptor by a subset of NK cells. A positive correlation between higher protection against allogeneic NK cell lysis and higher percentage of CD94/NKG2A positive NK cells was in fact observed (data not shown).

### HLA-I DEFICIENT NK-92 Cells do not Trigger Allogeneic CD8+ T Cell Responses

NK-92 cells trigger CD8+ or CD4+ T cell proliferation in standard mixed lymphocyte reaction (MLR) experiments (FIG. 1), indicating that these cells are immunogenic. In addition, antibodies against HLA molecules expressed by NK-92 cells have been detected in patients that have received infusions of NK-92 cells. Therefore, because current clinical protocols involve multiple infusions of irradiated NK-92 cells, there is a risk that some patients may mount an immune response against NK-92 cells and compromise effectiveness.

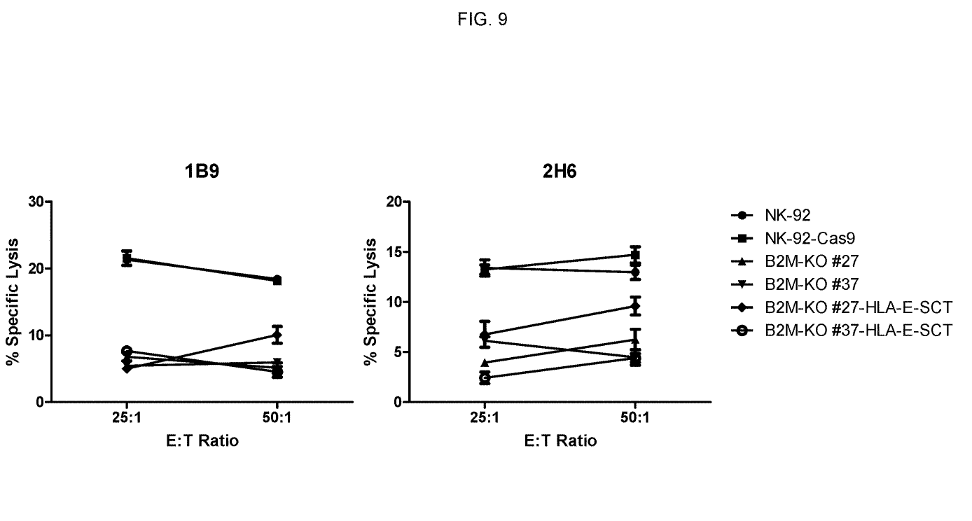

HLA-I deficient NK-92 cells should not be recognized by CD8+ T cells, since they lack classical HLA-A, -B, and -C molecules that present antigenic peptides to CD8+ T cells through binding to their TCRs (T cell receptors). To formally prove the lack of immunogenic potential of the HLA-I deficient NK-92 cells we generated polyclonal CD8+ T cells reactive against parental NK-92 cells (as described in Materials and Methods). Notably, these NK-92 specific CD8+ T cells were able to recognize and kill parental NK-92 cells, but failed to lyse HLA-I deficient NK-92 cells (FIG. 9).

### Materials and Methods

### Cell Culture

NK-92 cells were maintained in X-VIVO 10 medium (Lonza, catalog # BE04-743Q) supplemented with 5% Human Serum (Valley Biomedical, catalog # HP1022) and recombinant human IL-2 (500 IU/ml; Prospec, catalog # Cyt-209). K562 cells were purchased from American Type Culture Collection (ATCC, Rockville, Md.), and maintained in RPMI-1640 medium (Thermo Scientific, catalog #61870-127) supplemented with 10% FBS (Gibco, catalog #10438026) and 1% Penicillin/Streptomycin (Gibco, catalog #15070-063).

### HLA-E-SCT (Single Chain Trimer) Design and Sequences

The HLA-E single chain trimer (SCT) encompasses the following sequences: B2M (β2 microglobulin) signal peptide-Cw*0304 leader peptide-linker (G₄S)₃-mature B2M sequence-linker (G₄S)₄-mature HLA-E sequence (FIG. 6). The DNA and protein sequences of HLA-E-SCT correspond to:

### B2M Signal Peptide

B2M, beta-2-microglobulin→Gene ID: 567.

### Protein: UniProt P61769

B2M signal peptide amino acid sequence:
(SEQ ID NO: 8)
MSRSVALAVLALLSLSGLEA
B2M signal peptide nucleotide sequence:
(SEQ ID NO: 9)
ATGTCTCGCTCCGTGGCCTTAGCTGTGCTCGCGCTACTCTCTCTTTCTGG
CCTGGAGGCT

### Linkers

Linker (G₄S)₃ nucleotide sequence:
(SEQ ID NO: 10)
GGTGGCGGTGGCTCGGGCGGTGGTGGGTCGGGTGGCGGCGGATCT
Linker (G₄S)₄ nucleotide sequence:
(SEQ ID NO: 11)
GGAGGAGGTGGGTCTGGAGGTGGAGGATCTGGTGGAGGTGGGTCTGGAGG
AGGTGGGTCT

### Cw*0304 Peptide

The Cw*0304 peptide corresponds to the leader peptide of HLA class I histocompatibility antigen Cw-3 alpha chain (UniProt: P04222)

Amino acid sequence:
(SEQ ID NO: 12)
VMAPRTLIL
Nucleotide sequence encoding Cw*0304 peptide:
(SEQ ID NO: 13)
GTCATGGCGCCCCGAACCCTCATCCTG

### B2M Mature Chain

Amino acid sequence:
(SEQ ID NO: 14)
IQRTPKIQVYSRHPAENGKSNFLNCYVSGFHPSDIEVDLLKNGERIEKVE
HSDLSFSKDWSFYLLYYTEFTPTEKDEYACRVNHVTLSQPKIVKWDRDM
Nucleotide sequence emcpdomg B2M mature chain
polypeptide:
(SEQ ID NO: 15)
ATCCAGCGTACTCCAAAGATTCAGGTTTACTCACGTCATCCAGCAGAGAA
TGGAAAGTCAAATTTCCTGAATTGCTATGTGTCTGGGTTTCATCCATCCG
ACATTGAAGTTGACTTACTGAAGAATGGAGAGAGAATTGAAAAAGTGGAG
CATTCAGACTTGTCTTTCAGCAAGGACTGGTCTTTCTATCTCTTGTACTA
CACTGAATTCACCCCCACTGAAAAAGATGAGTATGCCTGCCGTGTGAACC
ATGTGACTTTGTCACAGCCCAAGATAGTTAAGTGGGATCGAGACATG

### HLA-E Mature Chain

HLA-E→Gene ID: 3133. mRNA accession number NM_005516

### Protein: UniProt P13747

The HLA-E mature chain does not contain the signal peptide (first 21 amino acids). It contains a Gly at position 107, which corresponds to HLA-E^{G} (E*0101 allele).

Amino acid sequence:
(SEQ ID NO: 16)
GSHSLKYFHTSVSRPGRGEPRFISVGYVDDTQFVRFDNDAASPRMVPRAP
WMEQEGSEYWDRETRSARDTAQIFRVNLRTLRGYYNQSEAGSHTLQWMHG
CELGPDGRFLRGYEQFAYDGKDYLTLNEDLRSWTAVDTAAQISEQKSNDA
SEAEHQRAYLEDTCVEWLHKYLEKGKETLLHLEPPKTHVTHHPISDHEAT
LRCWALGFYPAEITLTWQQDGEGHTQDTELVETRPAGDGTFQKWAAVVVP
SGEEQRYTCHVQHEGLPEPVTLRWKPASQPTIPIVGIIAGLVLLGSVVSG
AVVAAVIWRKKSSGGKGGSYSKAEWSDSAQGSESHSL
Nucleotide sequence that encodes HLA-E mature
polypeptide sequence of SEQ ID NO: 16:
(SEQ ID NO: 17)
GGCTCCCACTCCTTGAAGTATTTCCACACTTCCGTGTCCCGGCCCGGCCG
CGGGGAGCCCCGCTTCATCTCTGTGGGCTACGTGGACGACACCCAGTTCG
TGCGCTTCGACAACGACGCCGCGAGTCCGAGGATGGTGCCGCGGGCGCCG
TGGATGGAGCAGGAGGGGTCAGAGTATTGGGACCGGGAGACACGGAGCGC
CAGGGACACCGCACAGATTTTCCGAGTGAATCTGCGGACGCTGCGCGGCT
ACTACAATCAGAGCGAGGCCGGGTCTCACACCCTGCAGTGGATGCATGGC
TGCGAGCTGGGGCCCGACGGGCGCTTCCTCCGCGGGTATGAACAGTTCGC
CTACGACGGCAAGGATTATCTCACCCTGAATGAGGACCTGCGCTCCTGGA
CCGCGGTGGACACGGCGGCTCAGATCTCCGAGCAAAAGTCAAATGATGCC
TCTGAGGCGGAGCACCAGAGAGCCTACCTGGAAGACACATGCGTGGAGTG
GCTCCACAAATACCTGGAGAAGGGGAAGGAGACGCTGCTTCACCTGGAGC
CCCCAAAGACACACGTGACTCACCACCCCATCTCTGACCATGAGGCCACC
CTGAGGTGCTGGGCCCTGGGCTTCTACCCTGCGGAGATCACACTGACCTG
GCAGCAGGATGGGGAGGGCCATACCCAGGACACGGAGCTCGTGGAGACCA
GGCCTGCAGGGGATGGAACCTTCCAGAAGTGGGCAGCTGTGGTGGTGCCT
TCTGGAGAGGAGCAGAGATACACGTGCCATGTGCAGCATGAGGGGCTACC
CGAGCCCGTCACCCTGAGATGGAAGCCGGCTTCCCAGCCCACCATCCCCA
TCGTGGGCATCATTGCTGGCCTGGTTCTCCTTGGATCTGTGGTCTCTGGA
GCTGTGGTTGCTGCTGTGATATGGAGGAAGAAGAGCTCAGGTGGAAAAGG
AGGGAGCTACTCTAAGGCTGAGTGGAGCGACAGTGCCCAGGGGTCTGAGT
CTCACAGCTTG
Full length HLA-E-SCT amino acid sequence:
(SEQ ID NO: 18)
MSRSVALAVLALLSLSGLEAVMAPRTLILGGGGSGGGGSGGGGSIQRTPK
IQVYSRHPAENGKSNFLNCYVSGFHPSDIEVDLLKNGERIEKVEHSDLSF
SKDWSFYLLYYTEFTPTEKDEYACRVNHVTLSQPKIVKWDRDMGGGGSGG
GGSGGGGSGGGGSGSHSLKYFHTSVSRPGRGEPRFISVGYVDDTQFVRFD
NDAASPRMVPRAPWMEQEGSEYWDRETRSARDTAQIFRVNLRTLRGYYNQ
SEAGSHTLQWMHGCELGPDGRFLRGYEQFAYDGKDYLTLNEDLRSWTAVD
TAAQISEQKSNDASEAEHQRAYLEDTCVEWLHKYLEKGKETLLHLEPPKT
HVTHHPISDHEATLRCWALGFYPAEITLTWQQDGEGHTQDTELVETRPAG
DGTFQKWAAVVVPSGEEQRYTCHVQHEGLPEPVTLRWKPASQPTIPIVGI
IAGLVLLGSVVSGAVVAAVIWRKKSSGGKGGSYSKAEWSDSAQGSESHSL
Full length HLA-E-SCT DNA sequence encoding
polypeptide sequence of SEQ ID NO: 18:
(SEQ ID NO: 19)
ATGTCTCGCTCCGTGGCCTTAGCTGTGCTCGCGCTACTCTCTCTTTCTGG
CCTGGAGGCTGTCATGGCGCCCCGAACCCTCATCCTGGGTGGCGGTGGCT
CGGGCGGTGGTGGGTCGGGTGGCGGCGGATCTATCCAGCGTACTCCAAAG
ATTCAGGTTTACTCACGTCATCCAGCAGAGAATGGAAAGTCAAATTTCCT
GAATTGCTATGTGTCTGGGTTTCATCCATCCGACATTGAAGTTGACTTAC
TGAAGAATGGAGAGAGAATTGAAAAAGTGGAGCATTCAGACTTGTCTTTC
AGCAAGGACTGGTCTTTCTATCTCTTGTACTACACTGAATTCACCCCCAC
TGAAAAAGATGAGTATGCCTGCCGTGTGAACCATGTGACTTTGTCACAGC
CCAAGATAGTTAAGTGGGATCGAGACATGGGAGGAGGTGGGTCTGGAGGT
GGAGGATCTGGTGGAGGTGGGTCTGGAGGAGGTGGGTCTGGCTCCCACTC
CTTGAAGTATTTCCACACTTCCGTGTCCCGGCCCGGCCGCGGGGAGCCCC
GCTTCATCTCTGTGGGCTACGTGGACGACACCCAGTTCGTGCGCTTCGAC
AACGACGCCGCGAGTCCGAGGATGGTGCCGCGGGCGCCGTGGATGGAGCA
GGAGGGGTCAGAGTATTGGGACCGGGAGACACGGAGCGCCAGGGACACCG
CACAGATTTTCCGAGTGAATCTGCGGACGCTGCGCGGCTACTACAATCAG
AGCGAGGCCGGGTCTCACACCCTGCAGTGGATGCATGGCTGCGAGCTGGG
GCCCGACGGGCGCTTCCTCCGCGGGTATGAACAGTTCGCCTACGACGGCA
AGGATTATCTCACCCTGAATGAGGACCTGCGCTCCTGGACCGCGGTGGAC
ACGGCGGCTCAGATCTCCGAGCAAAAGTCAAATGATGCCTCTGAGGCGGA
GCACCAGAGAGCCTACCTGGAAGACACATGCGTGGAGTGGCTCCACAAAT
ACCTGGAGAAGGGGAAGGAGACGCTGCTTCACCTGGAGCCCCCAAAGACA
CACGTGACTCACCACCCCATCTCTGACCATGAGGCCACCCTGAGGTGCTG
GGCCCTGGGCTTCTACCCTGCGGAGATCACACTGACCTGGCAGCAGGATG
GGGAGGGCCATACCCAGGACACGGAGCTCGTGGAGACCAGGCCTGCAGGG
GATGGAACCTTCCAGAAGTGGGCAGCTGTGGTGGTGCCTTCTGGAGAGGA
GCAGAGATACACGTGCCATGTGCAGCATGAGGGGCTACCCGAGCCCGTCA
CCCTGAGATGGAAGCCGGCTTCCCAGCCCACCATCCCCATCGTGGGCATC
ATTGCTGGCCTGGTTCTCCTTGGATCTGTGGTCTCTGGAGCTGTGGTTGC
TGCTGTGATATGGAGGAAGAAGAGCTCAGGTGGAAAAGGAGGGAGCTACT
CTAAGGCTGAGTGGAGCGACAGTGCCCAGGGGTCTGAGTCTCACAGCTTG
TAA

### Lentivirus Production and Infection

The HLA-E-SCT gene was cloned into the lentiviral vector pCDH-EF1-MCS-PGK-Puro (System Biosciences, catalog # CD810A-1) using the restriction sites BamHI and SalI. HLA-E-SCT encoding lentivirus stocks were produced by transfecting 7×10⁶ 293 T cells per 10 cm petri dish with the following amount of plasmids: 7.5 μg pCDH-EF1-MCS-PGK-Puro lentiviral vector expressing HLA-E-SCT, 5 μg pCMV-AR8.2, and 2.5 μg pCMV-VSV.G. The transfections were performed using Lipofectamine 3000 (Life Technologies, catalog # L3000-008) following manufacturer's instructions. Virus supernatants were collected 48 h post-transfection, and concentrated 10 fold using PEG-it Virus Precipitation Solution from System Biosciences (catalog # LV810A-1).

Cell lines stably expressing HLA-E-SCT were generated by infecting HLA-I deficient NK-92 or K562 cells with HLA-E-SCT encoding lentivirus. In brief, 5×10⁵ NK-92 or K562 cells were infected by spinoculation (840 g for 99 min at 35° C.) with 100 μl of concentrated virus in 1 ml of final medium in a 24 well plate, in the presence of TransDux (System Biosciences, catalog # LV850A-1). Forty eight hours post-transduction, the HLA-E-SCT-expressing cells were selected by growing the cells in the presence of 2 μg/ml of puromycin (SIGMA, catalog # P9620).

### Flow Cytometry

Cytofluorometric analyses were performed on a MACSQuant 10 flow cytometer (Miltenyi) and analyzed using FlowJo software. Antibodies were purchased from BioLegend and include: anti-B2M-PE (cat #316306), anti-HLA-I-PE (cat #311406), anti-HLA-E-APC (cat #342606), anti-CD3-PE (cat #300408), anti-CD8-AF647 (cat #300918), IgG1-APC control (cat #400122), IgG1-AF647 control (cat #400136), and IgG1-PE control (cat #400114).

### Cytotoxicity Assays

Target cells were stained with the fluorescent dye PKH67-GL (Sigma-Aldrich, Saint Louis, Mo.) according to manufacturer's instructions. Targets and effectors were combined at different effector to target (E:T) ratios in a 96-well plate (Falcon BD, Franklin Lakes, N.J.), briefly centrifuged, and incubated in X-VIVO 10 (Lonza, cat #04-743Q) culture medium, supplemented with 5% human serum, at 37° C. for 4 h in a 5% CO₂ incubator. After incubation, cells were stained with propidium iodide (PI, Sigma-Aldrich) at 10 μg/ml in 1% BSA/PBS buffer and analyzed immediately by flow cytometry. Dead target cells were identified as double positive for PKH67-GL and PI. Target cells and effector cells were also stained separately with PI to assess spontaneous cell lysis. The percentage of NK-mediated cytotoxicity was obtained by subtracting the percentage of PKH(+)/PI(+) cells for target cells alone (spontaneous lysis) from the percentage of PKH(+)/PI(+) cells in the samples with effectors.

### NK Cell Purification

PBMCs from healthy donors were purified by ficoll hypaque gradient centrifugation using buffy coats purchased from Research Blood Components (website address http researchbloodcomponents.com). NK cells were purified using CD56 MicroBeads (Miltenyi, 130-050-401) and LS columns (Miltenyi, 130-042-401) following manufacturer's instructions. Purity of the CD56+/CD3− NK cells was verified by flow cytometry using anti-CD3-FITC (BD Pharmingen, cat #555332) and anti-CD56-PE (BD Pharmingen, cat #555516) antibodies, and were consistently ≥80% CD56+/CD3−. The purified NK cells were used either right after purification (non-activated NK cells) or grown in X-VIVO 10/5% Human Serum plus 10³ U/ml of IL2 for 6-9 days (activated NK cells).

### Generation of NK-92 Specific Allogeneic CD8+ T Cells

CD8+ T cells were purified from PBMCs using the CD8+ T Cell Isolation Kit from Miltenyi (cat #130-096-495) following manufacturer's instructions. Purity of the CD8+ T cells was verified by flow cytometry using anti-CD3-FITC (BD Pharmingen, cat #555332) and anti-CD8-AF647 (BioLegend, cat #300918) antibodies, and were consistently ≥80% CD3+/CD8+. To generate NK-92 specific allogeneic CD8+ T cells, 5×10⁴ purified CD8+ T cells were plated in “U” bottom 96 well plates with 5×10⁴ irradiated (10 Gy) NK-92 cells (1:1 ratio). Cells were plated in X-VIVO 10 medium supplemented with 5% Human Serum with no cytokines. CD8+ T cells were re-stimulated with freshly irradiated NK-92 cells after 9-12 days of culture. Wells that showed proliferation of stimulated CD8+ T cells were further expanded by growing the cells in X-VIVO 10 medium supplemented with 5% Human Serum and 0.5 μg/ml of PHA-L plus 500 IU/ml of IL-2.

It is understood that the examples and embodiments described herein are for illustrative purposes only and that various modifications or changes in light thereof will be suggested to persons skilled in the art and are to be included within the spirit and purview of this application and scope of the appended claims. All publications, sequence accession numbers, patents, and patent applications cited herein are hereby incorporated by reference in their entirety for all purposes.

TABLE OF ILLUSTRATIVE SEQUENCES

SEQ ID NO: 1 Guide RNA number 1 in Example 3
GAGTAGCGCGAGCACAGCTA
SEQ ID NO: 2 Guide RNA number 2 in Example 3
CGCGAGCACAGCTAAGGCCA
SEQ ID NO: 3 Guide RNA number 3 in Example 3
CTCGCGCTACTCTCTCTTTC
SEQ ID NO: 4 Guide RNA number 4 in Example 3
GCTACTCTCTCTTTCTGGCC
SEQ ID NO: 5 CD 16 High Affinity Variant F158V Immunoglobulin Gamma Fc Region
Receptor III-A amino acid sequence (mature form):
Arg Thr Glu Asp Leu Pro Lys Ala Val Val Phe Leu Glu Pro Gln Trp Tyr Arg Val Leu Glu
Lys Asp Ser Val Thr Leu Lys Cys Gln Gly Ala Tyr Ser Pro Glu Asp Asn Ser Thr Gln Trp
Phe His Asn Glu Ser Leu Ile Ser Ser Gln Ala Ser Ser Tyr Phe Ile Asp Ala Ala Thr Val Asp
Asp Ser Gly Glu Tyr Arg Cys Gln Thr Asn Leu Ser Thr Leu Ser Asp Pro Val Gln Leu Glu
Val His Ile Gly Trp Leu Leu Leu Gln Ala Pro Arg Trp Val Phe Lys Glu Glu Asp Pro Ile His
Leu Arg Cys His Ser Trp Lys Asn Thr Ala Leu His Lys Val Thr Tyr Leu Gln Asn Gly Lys
Gly Arg Lys Tyr Phe His His Asn Ser Asp Phe Tyr Ile Pro Lys Ala Thr Leu Lys Asp Ser Gly
Ser Tyr Phe Cys Arg Gly Leu Val Gly Ser Lys Asn Val Ser Ser Glu Thr Val Asn Ile Thr Ile
Thr Gln Gly Leu Ala Val Ser Thr Ile Ser Ser Phe Phe Pro Pro Gly Tyr Gln Val Ser Phe Cys
Leu Val Met Val Leu Leu Phe Ala Val Asp Thr Gly Leu Tyr Phe Ser Val Lys Thr Asn Ile
Arg Ser Ser Thr Arg Asp Trp Lys Asp His Lys Phe Lys Trp Arg Lys Asp Pro Gln Asp Lys
SEQ ID NO: 6 human beta-2-microglobulin (B2M) precursor polypeptide sequence
(NP_004039)
MSRSVALAVLALLSLSGLEAIQRTPKIQVYSRHPAENGKSNFLNCYVSGFHPSDIEVD
LLKNGERIEKVEHSDLSFSKDWSFYLLYYTEFTPTEKDEYACRVNHVTLSQPKIVKW
DRDM
SEQ ID NO: 7 human HLA-E sequence encoded by accession number NM_005516
MVDGTLLLLLSEALALTQTWAGSHSLKYFHTSVSRPGRGEPRFISVGYVDDTQFVRF
DNDAASPRMVPRAPWMEQEGSEYWDRETRSARDTAQIFRVNLRTLRGYYNQSEAG
SHTLQWMHGCELGPDGRFLRGYEQFAYDGKDYLTLNEDLRSWTAVDTAAQISEQK
SNDASEAEHQRAYLEDTCVEWLHKYLEKGKETLLHLEPPKTHVTHHPISDHEATLRC
WALGFYPAEITLTWQQDGEGHTQDTELVETRPAGDGTFQKWAAVVVPSGEEQRYT
CHVQHEGLPEPVTLRWKPASQPTIPIVGIIAGLVLLGSVVSGAVVAAVIWRKKSSGGK
GGSYSKAEWSDSAQGSESHSL
SEQ ID NO: 8 B2M signal peptide amino acid sequence
MSRSVALAVLALLSLSGLEA
SEQ ID NO: 9 B2M signal peptide nucleotide sequence
ATGTCTCGCTCCGTGGCCTTAGCTGTGCTCGCGCTACTCTCTCTTTCTGGCCTGGA
GGCT
SEQ ID NO: 10 Linker (G₄S)₃ nucleotide sequence
GGTGGCGGTGGCTCGGGCGGTGGTGGGTCGGGTGGCGGCGGATCT
SEQ ID No: 11 Linker (G₄S)₄ nucleotide sequence
GGAGGAGGTGGGTCTGGAGGTGGAGGATCTGGTGGAGGTGGGTCTGGAGGAGGT
GGGTCT
SEQ ID NO: 12 Cw*0304 peptide, which corresponds to the leader peptide of HLA class
I histocompatibility antigen Cw-3 alpha chain (UniProt: P04222), amino acid sequence
VMAPRTLIL
SEQ ID NO: 13 Nucleotide sequence encoding Cw*0304 peptide of SEQ ID NO: 12
GTCATGGCGCCCCGAACCCTCATCCTG
SEQ ID NO: 14 B2M mature chain amino acid sequence
IQRTPKIQVYSRHPAENGKSNFLNCYVSGFHPSDIEVDLLKNGERIEKVEHSDLSFSKD
WSFYLLYYTEFTPTEKDEYACRVNHVTLSQPKIVKWDRDM
SEQ ID NO: 15 Nucleic acid sequence encoding B2M mature chain amino acid
sequence
ATCCAGCGTACTCCAAAGATTCAGGTTTACTCACGTCATCCAGCAGAGAATGGAA
AGTCAAATTTCCTGAATTGCTATGTGTCTGGGTTTCATCCATCCGACATTGAAGTT
GACTTACTGAAGAATGGAGAGAGAATTGAAAAAGTGGAGCATTCAGACTTGTCT
TTCAGCAAGGACTGGTCTTTCTATCTCTTGTACTACACTGAATTCACCCCCACTGA
AAAAGATGAGTATGCCTGCCGTGTGAACCATGTGACTTTGTCACAGCCCAAGATA
GTTAAGTGGGATCGAGACATG
SEQ ID NO: 16 HLA-E mature polypeptide sequence lacking the signal peptide HLA-E^{G}
(E*0101 allele)
GSHSLKYFHTSVSRPGRGEPRFISVGYVDDTQFVRFDNDAASPRMVPRAPWMEQEG
SEYWDRETRSARDTAQIFRVNLRTLRGYYNQSEAGSHTLQWMHGCELGPDGRFLRG
YEQFAYDGKDYLTLNEDLRSWTAVDTAAQISEQKSNDASEAEHQRAYLEDTCVEW
LHKYLEKGKETLLHLEPPKTHVTHHPISDHEATLRCWALGFYPAEITLTWQQDGEGH
TQDTELVETRPAGDGTFQKWAAVVVPSGEEQRYTCHVQHEGLPEPVTLRWKPASQP
TIPIVGIIAGLVLLGSVVSGAVVAAVIWRKKSSGGKGGSYSKAEWSDSAQGSESHSL
SEQ ID NO: 17 Nucleic acid sequence encoding SEQ ID NO: 16
GGCTCCCACTCCTTGAAGTATTTCCACACTTCCGTGTCCCGGCCCGGCCGCGGGG
AGCCCCGCTTCATCTCTGTGGGCTACGTGGACGACACCCAGTTCGTGCGCTTCGA
CAACGACGCCGCGAGTCCGAGGATGGTGCCGCGGGCGCCGTGGATGGAGCAGGA
GGGGTCAGAGTATTGGGACCGGGAGACACGGAGCGCCAGGGACACCGCACAGA
TTTTCCGAGTGAATCTGCGGACGCTGCGCGGCTACTACAATCAGAGCGAGGCCG
GGTCTCACACCCTGCAGTGGATGCATGGCTGCGAGCTGGGGCCCGACGGGCGCT
TCCTCCGCGGGTATGAACAGTTCGCCTACGACGGCAAGGATTATCTCACCCTGAA
TGAGGACCTGCGCTCCTGGACCGCGGTGGACACGGCGGCTCAGATCTCCGAGCA
AAAGTCAAATGATGCCTCTGAGGCGGAGCACCAGAGAGCCTACCTGGAAGACAC
ATGCGTGGAGTGGCTCCACAAATACCTGGAGAAGGGGAAGGAGACGCTGCTTCA
CCTGGAGCCCCCAAAGACACACGTGACTCACCACCCCATCTCTGACCATGAGGCC
ACCCTGAGGTGCTGGGCCCTGGGCTTCTACCCTGCGGAGATCACACTGACCTGGC
AGCAGGATGGGGAGGGCCATACCCAGGACACGGAGCTCGTGGAGACCAGGCCT
GCAGGGGATGGAACCTTCCAGAAGTGGGCAGCTGTGGTGGTGCCTTCTGGAGAG
GAGCAGAGATACACGTGCCATGTGCAGCATGAGGGGCTACCCGAGCCCGTCACC
CTGAGATGGAAGCCGGCTTCCCAGCCCACCATCCCCATCGTGGGCATCATTGCTG
GCCTGGTTCTCCTTGGATCTGTGGTCTCTGGAGCTGTGGTTGCTGCTGTGATATGG
AGGAAGAAGAGCTCAGGTGGAAAAGGAGGGAGCTACTCTAAGGCTGAGTGGAG
CGACAGTGCCCAGGGGTCTGAGTCTCACAGCTTG
SEQ ID NO: 18 Full length HLA-E-SCT amino acid sequence:
MSRSVALAVLALLSLSGLEAVMAPRTLILGGGGSGGGGSGGGGSIQRTPKIQVYSRH
PAENGKSNFLNCYVSGFHPSDIEVDLLKNGERIEKVEHSDLSFSKDWSFYLLYYTEFT
PTEKDEYACRVNHVTLSQPKIVKWDRDMGGGGSGGGGSGGGGSGGGGSGSHSLKY
FHTSVSRPGRGEPRFISVGYVDDTQFVRFDNDAASPRMVPRAPWMEQEGSEYWDRE
TRSARDTAQIFRVNLRTLRGYYNQSEAGSHTLQWMHGCELGPDGRFLRGYEQFAYD
GKDYLTLNEDLRSWTAVDTAAQISEQKSNDASEAEHQRAYLEDTCVEWLHKYLEK
GKETLLHLEPPKTHVTHHPISDHEATLRCWALGFYPAEITLTWQQDGEGHTQDTELV
ETRPAGDGTFQKWAAVVVPSGEEQRYTCHVQHEGLPEPVTLRWKPASQPTIPIVGIIA
GLVLLGSVVSGAVVAAVIWRKKSSGGKGGSYSKAEWSDSAQGSESHSL
SEQ ID NO: 19 Nucleic acid sequence encoding SEQ ID NO: 18
ATGTCTCGCTCCGTGGCCTTAGCTGTGCTCGCGCTACTCTCTCTTTCTGGCCTGGA
GGCTGTCATGGCGCCCCGAACCCTCATCCTGGGTGGCGGTGGCTCGGGCGGTGGT
GGGTCGGGTGGCGGCGGATCTATCCAGCGTACTCCAAAGATTCAGGTTTACTCAC
GTCATCCAGCAGAGAATGGAAAGTCAAATTTCCTGAATTGCTATGTGTCTGGGTT
TCATCCATCCGACATTGAAGTTGACTTACTGAAGAATGGAGAGAGAATTGAAAA
AGTGGAGCATTCAGACTTGTCTTTCAGCAAGGACTGGTCTTTCTATCTCTTGTACT
ACACTGAATTCACCCCCACTGAAAAAGATGAGTATGCCTGCCGTGTGAACCATGT
GACTTTGTCACAGCCCAAGATAGTTAAGTGGGATCGAGACATGGGAGGAGGTGG
GTCTGGAGGTGGAGGATCTGGTGGAGGTGGGTCTGGAGGAGGTGGGTCTGGCTC
CCACTCCTTGAAGTATTTCCACACTTCCGTGTCCCGGCCCGGCCGCGGGGAGCCC
CGCTTCATCTCTGTGGGCTACGTGGACGACACCCAGTTCGTGCGCTTCGACAACG
ACGCCGCGAGTCCGAGGATGGTGCCGCGGGCGCCGTGGATGGAGCAGGAGGGGT
CAGAGTATTGGGACCGGGAGACACGGAGCGCCAGGGACACCGCACAGATTTTCC
GAGTGAATCTGCGGACGCTGCGCGGCTACTACAATCAGAGCGAGGCCGGGTCTC
ACACCCTGCAGTGGATGCATGGCTGCGAGCTGGGGCCCGACGGGCGCTTCCTCC
GCGGGTATGAACAGTTCGCCTACGACGGCAAGGATTATCTCACCCTGAATGAGG
ACCTGCGCTCCTGGACCGCGGTGGACACGGCGGCTCAGATCTCCGAGCAAAAGT
CAAATGATGCCTCTGAGGCGGAGCACCAGAGAGCCTACCTGGAAGACACATGCG
TGGAGTGGCTCCACAAATACCTGGAGAAGGGGAAGGAGACGCTGCTTCACCTGG
AGCCCCCAAAGACACACGTGACTCACCACCCCATCTCTGACCATGAGGCCACCCT
GAGGTGCTGGGCCCTGGGCTTCTACCCTGCGGAGATCACACTGACCTGGCAGCA
GGATGGGGAGGGCCATACCCAGGACACGGAGCTCGTGGAGACCAGGCCTGCAG
GGGATGGAACCTTCCAGAAGTGGGCAGCTGTGGTGGTGCCTTCTGGAGAGGAGC
AGAGATACACGTGCCATGTGCAGCATGAGGGGCTACCCGAGCCCGTCACCCTGA
GATGGAAGCCGGCTTCCCAGCCCACCATCCCCATCGTGGGCATCATTGCTGGCCT
GGTTCTCCTTGGATCTGTGGTCTCTGGAGCTGTGGTTGCTGCTGTGATATGGAGG
AAGAAGAGCTCAGGTGGAAAAGGAGGGAGCTACTCTAAGGCTGAGTGGAGCGA
CAGTGCCCAGGGGTCTGAGTCTCACAGCTTGTAA
SEQ ID NO: 20 leader amino acid sequence of HLA-A*0201
VMAPRTLVL

1. A beta-2-microglobulin-modified (B2M-modified) NK-92 cell comprising a beta-2 microglobulin-targeted genetic modification to inhibit expression of beta-2 microglobulin.

2. The B2M-modified NK-92 cell of claim 1, wherein the cell is produced by knocking down or knocking out beta-2 microglobulin in an NK-92 cell.

3. The B2M-modified NK-92 cells of claim 2, comprising an interfering RNA that targets B2M and inhibits its expression.

4.-5. (canceled)

6. The B2M-modified NK-92 cell of claim 2, wherein the cell is modified to express a single chain trimer comprising an HLA-E binding peptide, B2M, and HLA-E heavy chain.

7. The B2M-modified NK-92 cell of claim 6, wherein the single chain trimer comprises a B2M (β2 microglobulin) signal peptide, a Cw*0304 leader peptide, a mature B2M polypeptide and a mature HLA-E polypeptide.

8. The B2M-modified NK-92 cell of claim 7, wherein the Cw*0304 leader peptide is linked to the mature B2M polypeptide by a flexible linker and/or the mature B2M polypeptide is linked to the mature HLA-E polypeptide by a flexible linker.

9. (canceled)

10. The B2M-modified NK-92 cell of claim 6, wherein the HLA-E heavy chain comprises a mature HLA-EG amino acid sequence.

11. The B2M-modified NK-92 cell of claim 6, wherein the single chain trimer comprises the amino acid sequence of SEQ ID NO:18.

12. The B2M-modified NK-92 cell of claim 1, wherein the B2M-modified NK cell expresses at least one Fc receptor or at least one chimeric antigen receptor (CAR); or at least one Fc receptor and at least one CAR on the cell surface.

13. The B2M-modified NK-92 cell of claim 12, wherein the at least one Fc receptor is a human CD16 or a human CD16 polypeptide having a valine at a position corresponding to position 158 of the mature form of the CD16 polypeptide.

14.-17. (canceled)

18. The B2M-modified NK-92 cell of claim 1, wherein the cell is further modified to expresses a cytokine.

19.-20. (canceled)

21. A composition comprising a plurality of cells of claim 1.

22. (canceled)

23. A modified NK-92 cell line comprising a plurality of modified NK-92 cells of claim 1.

24. The cell line of claim 23, wherein the cells undergo less than 10 population doublings.

25. (canceled)

26. A method of treating cancer in a patient in need thereof, the method comprising administering to the patient a therapeutically effective amount of the cell line of claim 23, thereby treating the cancer.

27.-28. (canceled)

29. A method for producing an NK-92 cell that expresses decreased levels of beta-2 microglobulin relative to a control NK-92 cell, the method comprising genetically modifying the NK-92 cell to inhibit beta-2 microglobulin expression.

30. The method of claim 29, wherein the step of genetically modifying beta-2 microglobulin expression comprises modifying the beta-2 microglobulin gene with a zinc finger nuclease (ZFN), a Tale-effector domain nuclease (TALEN), or a CRIPSR/Cas system to eliminate or reduce expression of the beta-2 microglobulin gene; or comprises contacting a NK-92 cell to be modified with an interfering RNA targeting beta-2 microglobulin.

31. The method of claim 30, wherein the step of genetically modifying beta-2 microglobulin expression comprises modifying the beta-2 microglobulin gene with a CRIPSR/Cas system to eliminate or reduce expression of the beta-2 microglobulin gene.

32.-34. (canceled)

35. The method of claim 31, wherein genetically modifying the beta-2 microglobulin gene expression comprises:
i) introducing a clustered regularly interspaced short palindromic repeat-associated (Cas) protein into the NK-92 cell and
ii) introducing one or more ribonucleic acids in the NK-92 cell to be modified, wherein the ribonucleic acids direct the Cas protein to hybridize to a target motif of the beta-2 microglobulin sequence, and wherein the target motif is cleaved.

36.-38. (canceled)

39. The method of claim 35, wherein the target motif is in the first exon of beta 2 microglobulin gene.

40.-41. (canceled)


SEQUENCE LISTING (20 sequences)

SEQ ID NO: 1 - DNA, 20 bp, Artificial Sequence
  Synthteic construct
gagtagcgcgagcacagcta

SEQ ID NO: 2 - DNA, 20 bp, Artificial Sequence
  Synthteic construct
cgcgagcacagctaaggcca

SEQ ID NO: 3 - DNA, 20 bp, Artificial Sequence
  Synthteic construct
ctcgcgctactctctctttc

SEQ ID NO: 4 - DNA, 20 bp, Artificial Sequence
  Synthteic construct
gctactctctctttctggcc

SEQ ID NO: 5 - protein, 236 aa, Homo sapiens
RTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQVSFCLVMVLLFAVDTGLYFSVKTNIRSSTRDWKDHKFKWRKDPQDK

SEQ ID NO: 6 - protein, 119 aa, Homo sapiens
MSRSVALAVLALLSLSGLEAIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 7 - protein, 358 aa, Homo sapiens
MVDGTLLLLLSEALALTQTWAGSHSLKYFHTSVSRPGRGEPRFISVGYVDDTQFVRFDNDAASPRMVPRAPWMEQEGSEYWDRETRSARDTAQIFRVNLRTLRGYYNQSEAGSHTLQWMHGCELGPDGRFLRGYEQFAYDGKDYLTLNEDLRSWTAVDTAAQISEQKSNDASEAEHQRAYLEDTCVEWLHKYLEKGKETLLHLEPPKTHVTHHPISDHEATLRCWALGFYPAEITLTWQQDGEGHTQDTELVETRPAGDGTFQKWAAVVVPSGEEQRYTCHVQHEGLPEPVTLRWKPASQPTIPIVGIIAGLVLLGSVVSGAVVAAVIWRKKSSGGKGGSYSKAEWSDSAQGSESHSL

SEQ ID NO: 8 - protein, 20 aa, Homo sapiens
MSRSVALAVLALLSLSGLEA

SEQ ID NO: 9 - DNA, 60 bp, Homo sapiens
atgtctcgctccgtggccttagctgtgctcgcgctactctctctttctggcctggaggct

SEQ ID NO: 10 - DNA, 45 bp, Artificial Sequence
  Synthteic construct
ggtggcggtggctcgggcggtggtgggtcgggtggcggcggatct

SEQ ID NO: 11 - DNA, 60 bp, Artificial Sequence
  Synthteic construct
ggaggaggtgggtctggaggtggaggatctggtggaggtgggtctggaggaggtgggtct

SEQ ID NO: 12 - protein, 9 aa, Homo sapiens
VMAPRTLIL

SEQ ID NO: 13 - DNA, 27 bp, Homo sapiens
gtcatggcgccccgaaccctcatcctg

SEQ ID NO: 14 - protein, 99 aa, Homo sapiens [sequence omitted]

SEQ ID NO: 15 - DNA, 297 bp, Homo sapiens
atccagcgtactccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtgggatcgagacatg

SEQ ID NO: 16 - protein, 337 aa, Homo sapiens
GSHSLKYFHTSVSRPGRGEPRFISVGYVDDTQFVRFDNDAASPRMVPRAPWMEQEGSEYWDRETRSARDTAQIFRVNLRTLRGYYNQSEAGSHTLQWMHGCELGPDGRFLRGYEQFAYDGKDYLTLNEDLRSWTAVDTAAQISEQKSNDASEAEHQRAYLEDTCVEWLHKYLEKGKETLLHLEPPKTHVTHHPISDHEATLRCWALGFYPAEITLTWQQDGEGHTQDTELVETRPAGDGTFQKWAAVVVPSGEEQRYTCHVQHEGLPEPVTLRWKPASQPTIPIVGIIAGLVLLGSVVSGAVVAAVIWRKKSSGGKGGSYSKAEWSDSAQGSESHSL

SEQ ID NO: 17 - DNA, 1011 bp, Homo sapiens
ggctcccactccttgaagtatttccacacttccgtgtcccggcccggccgcggggagccccgcttcatctctgtgggctacgtggacgacacccagttcgtgcgcttcgacaacgacgccgcgagtccgaggatggtgccgcgggcgccgtggatggagcaggaggggtcagagtattgggaccgggagacacggagcgccagggacaccgcacagattttccgagtgaatctgcggacgctgcgcggctactacaatcagagcgaggccgggtctcacaccctgcagtggatgcatggctgcgagctggggcccgacgggcgcttcctccgcgggtatgaacagttcgcctacgacggcaaggattatctcaccctgaatgaggacctgcgctcctggaccgcggtggacacggcggctcagatctccgagcaaaagtcaaatgatgcctctgaggcggagcaccagagagcctacctggaagacacatgcgtggagtggctccacaaatacctggagaaggggaaggagacgctgcttcacctggagcccccaaagacacacgtgactcaccaccccatctctgaccatgaggccaccctgaggtgctgggccctgggcttctaccctgcggagatcacactgacctggcagcaggatggggagggccatacccaggacacggagctcgtggagaccaggcctgcaggggatggaaccttccagaagtgggcagctgtggtggtgccttctggagaggagcagagatacacgtgccatgtgcagcatgaggggctacccgagcccgtcaccctgagatggaagccggcttcccagcccaccatccccatcgtgggcatcattgctggcctggttctccttggatctgtggtctctggagctgtggttgctgctgtgatatggaggaagaagagctcaggtggaaaaggagggagctactctaaggctgagtggagcgacagtgcccaggggtctgagtctcacagcttg

SEQ ID NO: 18 - protein, 500 aa, Homo sapiens
MSRSVALAVLALLSLSGLEAVMAPRTLILGGGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGGGGSGSHSLKYFHTSVSRPGRGEPRFISVGYVDDTQFVRFDNDAASPRMVPRAPWMEQEGSEYWDRETRSARDTAQIFRVNLRTLRGYYNQSEAGSHTLQWMHGCELGPDGRFLRGYEQFAYDGKDYLTLNEDLRSWTAVDTAAQISEQKSNDASEAEHQRAYLEDTCVEWLHKYLEKGKETLLHLEPPKTHVTHHPISDHEATLRCWALGFYPAEITLTWQQDGEGHTQDTELVETRPAGDGTFQKWAAVVVPSGEEQRYTCHVQHEGLPEPVTLRWKPASQPTIPIVGIIAGLVLLGSVVSGAVVAAVIWRKKSSGGKGGSYSKAEWSDSAQGSESHSL

SEQ ID NO: 19 - DNA, 1503 bp, Homo sapiens
atgtctcgctccgtggccttagctgtgctcgcgctactctctctttctggcctggaggctgtcatggcgccccgaaccctcatcctgggtggcggtggctcgggcggtggtgggtcgggtggcggcggatctatccagcgtactccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtgggatcgagacatgggaggaggtgggtctggaggtggaggatctggtggaggtgggtctggaggaggtgggtctggctcccactccttgaagtatttccacacttccgtgtcccggcccggccgcggggagccccgcttcatctctgtgggctacgtggacgacacccagttcgtgcgcttcgacaacgacgccgcgagtccgaggatggtgccgcgggcgccgtggatggagcaggaggggtcagagtattgggaccgggagacacggagcgccagggacaccgcacagattttccgagtgaatctgcggacgctgcgcggctactacaatcagagcgaggccgggtctcacaccctgcagtggatgcatggctgcgagctggggcccgacgggcgcttcctccgcgggtatgaacagttcgcctacgacggcaaggattatctcaccctgaatgaggacctgcgctcctggaccgcggtggacacggcggctcagatctccgagcaaaagtcaaatgatgcctctgaggcggagcaccagagagcctacctggaagacacatgcgtggagtggctccacaaatacctggagaaggggaaggagacgctgcttcacctggagcccccaaagacacacgtgactcaccaccccatctctgaccatgaggccaccctgaggtgctgggccctgggcttctaccctgcggagatcacactgacctggcagcaggatggggagggccatacccaggacacggagctcgtggagaccaggcctgcaggggatggaaccttccagaagtgggcagctgtggtggtgccttctggagaggagcagagatacacgtgccatgtgcagcatgaggggctacccgagcccgtcaccctgagatggaagccggcttcccagcccaccatccccatcgtgggcatcattgctggcctggttctccttggatctgtggtctctggagctgtggttgctgctgtgatatggaggaagaagagctcaggtggaaaaggagggagctactctaaggctgagtggagcgacagtgcccaggggtctgagtctcacagcttgtaa

SEQ ID NO: 20 - protein, 9 aa, Homo sapiens
VMAPRTLVL